(19) United States
(12) Patent Application Publication
(10) Pub. No.: US 2026/0200097 A1
(43) Pub. Date: Jul. 16, 2026

(54) METHODS AND SYSTEMS FOR POSITIONING ROBOTS AND ADJUSTING POSTURES

(71) Applicant: WUHAN UNITED IMAGING SURGICAL CO., LTD., Wuhan (CN)

(72) Inventors: Xiaoxue WANG, Wuhan (CN); Tong WU, Wuhan (CN); Xu ZHANG, Wuhan (CN); Junyu ZOU, Wuhan (CN); Jingyuan LIU, Wuhan (CN); Yong DENG, Wuhan (CN); Quanquan WANG, Wuhan (CN); Bo WU, Wuhan (CN); Qin HUANG, Wuhan (CN)

(73) Assignee: WUHAN UNITED IMAGING SURGICAL CO., LTD., Wuhan (CN)

(21) Appl. No.: 19/565,641

(22) Filed: Mar. 13, 2026

Related U.S. Application Data
(63) Continuation-in-part of application No. 18/506,980, filed on Nov. 10, 2023, now Pat. No. 12,583,124.
Application No. 18/506,980 is a continuation of application No. PCT/CN2022/092003, filed on May 10, 2022.

(30) Foreign Application Priority Data
May 10, 2021 (CN) 202110505732.6
Nov. 19, 2021 (CN) 202111400891.6

(51) Int. Cl. B25J 9/16

(52) U.S. Cl. CPC B25J 9/1697; B25J 9/1664

(57) ABSTRACT

Embodiments of the present disclosure provide a method and system for positioning a robot and adjusting a posture. The method may include obtaining a first image and a second image of a target object. The first image may be captured using an image capturing apparatus, and the second image may be captured using a medical imaging device. The method may also include determining at least one target region corresponding to at least one target portion of the target object from the first image. The at least one target portion may be less affected by physiological motions than other portions. The method may further include determining positioning information of the robot based on the at least one target region and the second image.

### CROSS-REFERENCE TO RELATED APPLICATIONS

This application is a continuation-in-part of U.S. application Ser. No. 18/506,980, filed on Nov. 10, 2023, which is a continuation of International Application No. PCT/CN2022/092003, filed on May 10, 2022, which claims priority to Chinese Patent Application No. 202110505732.6, filed on May 10, 2021, titled “METHODS, APPARATUS, SYSTEMS, AND COMPUTER DEVICES FOR POSITIONING ROBOTS,” and Chinese Patent Application No. 202111400891.6, filed on Nov. 19, 2021, titled “METHODS, SYSTEMS, AND STORAGE MEDIA FOR ADJUSTING POSTURES OF CAMERAS AND SPATIAL REGISTRATION,” the entire contents of each of which are hereby incorporated by reference.

### TECHNICAL FIELD

The present disclosure relates to the field of robots, and in particular, to methods and systems for positioning robots and adjusting postures.

### BACKGROUND

In recent years, robots are widely used in the medical field, such as orthopedics, neurosurgery, thoracoabdominal interventional surgeries or treatment, etc. Generally speaking, a robot includes a robotic arm with a multi-degree-of-freedom structure, which includes a base joint where a base of the robotic arm is located and an end joint where a flange of the robotic arm is located. The flange of the robotic arm is fixedly connected with end tools, such as surgical tools (e.g., electrode needles, puncture needles, syringes, ablation needles, etc.).

When the robots are used, it is necessary to precisely position the robots and adjust postures of the robots, such that preoperative planning and/or surgical operations can be performed accurately.

### SUMMARY

One embodiment of the present disclosure provides a method for positioning a robot. The method may include obtaining a first image and a second image of a target object, the first image being captured using an image capturing apparatus, and the second image being captured using a medical imaging device; determining at least one target region corresponding to at least one target portion of the target object from the first image, wherein the at least one target portion is less affected by physiological motions than other portions; and determining positioning information of the robot based on the at least one target region and the second image.

One embodiment of the present disclosure provides a method for adjusting a posture of an image capturing apparatus. The method may include capturing a target image of a target object using the image capturing apparatus; determining at least one target feature point of the target object from the target image; determining at least one reference feature point corresponding to the at least one target feature point from a reference model of the target object, wherein the reference model corresponds to a target shooting angle; and determining a first target posture of the image capturing apparatus in a base coordinate system based on the at least one target feature point and the at least one reference feature point.

One embodiment of the present disclosure provides a system for positioning a robot. The system may include a storage device configured to store a computer instruction, and a processor connected to the storage device. When executing the computer instruction, the processor may cause the system to perform the following operations: obtaining a first image and a second image of a target object, the first image being captured using an image capturing apparatus, and the second image being captured using a medical imaging device; determining at least one target region corresponding to at least one target portion of the target object from the first image, wherein the at least one target portion is less affected by physiological motions than other portions; and determining positioning information of the robot based on the at least one target region and the second image.

### BRIEF DESCRIPTION OF THE DRAWINGS

The present disclosure will be further illustrated by way of exemplary embodiments, which will be described in detail through the accompanying drawings. These embodiments are not limiting, and in these embodiments the same numbering indicates the same structure, wherein:

FIG. 1A is a schematic diagram illustrating an exemplary application scenario of a robotic control system according to some embodiments of the present disclosure;

FIG. 1B is a schematic diagram illustrating another exemplary application scenario of the robotic control system according to some embodiments of the present disclosure;

FIG. 1C is a schematic diagram illustrating an exemplary server according to some embodiments of the present disclosure;

FIG. 2 is a block diagram illustrating an exemplary processor according to some embodiments of the present disclosure;

FIG. 3 is a flowchart illustrating an exemplary process for robot positioning according to some embodiments of the present disclosure;

FIG. 4 is a flowchart illustrating an exemplary process for determining at least one target region according to some embodiments of the present disclosure;

FIG. 5 is a flowchart illustrating an exemplary process for determining a registration relationship according to some embodiments of the present disclosure;

FIG. 6A is a schematic diagram illustrating an exemplary reference point set in a second image according to some embodiments of the present disclosure;

FIG. 6B is a schematic diagram illustrating an exemplary candidate target point set in at least one target region according to some embodiments of the present disclosure;

FIG. 7 is a schematic diagram illustrating exemplary facial feature points according to some embodiments of the present disclosure;

FIG. 8 is a schematic diagram illustrating an exemplary two-dimensional facial image according to some embodiments of the present disclosure;

FIG. 9 is a block diagram illustrating an exemplary processor according to some embodiments of the present disclosure;

FIG. 10 is a flowchart illustrating an exemplary process for adjusting a posture of an image capturing apparatus according to some embodiments of the present disclosure;

FIG. 11 is a flowchart illustrating an exemplary process for determining a first target posture according to some embodiments of the present disclosure;

FIG. 12 is a flowchart illustrating an exemplary process for adjusting a posture of an image capturing apparatus according to some embodiments of the present disclosure;

FIG. 13A is a front view of an exemplary standard facial model according to some embodiments of the present disclosure;

FIG. 13B is a side view of an exemplary standard facial model according to some embodiments of the present disclosure;

FIG. 14 is a schematic diagram illustrating exemplary facial contour points according to some embodiments of the present disclosure;

FIGS. 15A to 15C are schematic diagrams illustrating an exemplary process for adjusting a posture of an image capturing apparatus according to some embodiments of the present disclosure;

FIG. 16A is a schematic diagram illustrating an image capturing apparatus before posture adjustment according to some embodiments of the present disclosure;

FIG. 16B is a schematic diagram illustrating an image capturing apparatus after posture adjustment according to some embodiments of the present disclosure;

FIGS. 16C, 16E, and 16G are schematic diagrams illustrating image data captured by an image capturing apparatus before posture adjustment according to some embodiments of the present disclosure;

FIGS. 16D, 16F, and 16H are schematic diagrams illustrating image data captured by an image capturing apparatus after posture adjustment according to some embodiments of the present disclosure.

FIG. 17 is a schematic diagram illustrating an exemplary posture adjustment process of an image capturing apparatus according to some embodiments of the present disclosure;

FIG. 18 is a schematic diagram illustrating an exemplary robotic control system according to some embodiments of the present disclosure;

FIG. 19 is a schematic diagram illustrating an exemplary computer device according to some embodiments of the present disclosure;

FIG. 20 is a flowchart illustrating an exemplary robotic control process according to some embodiments of the present disclosure;

FIG. 21 is a flowchart illustrating an exemplary process for robot positioning according to some embodiments of the present disclosure;

FIG. 22 is a flowchart illustrating an exemplary process for determining positioning information of a robot according to some embodiments of the present disclosure;

FIG. 23 is a schematic diagram illustrating an exemplary guidance image according to some embodiments of the present disclosure;

FIG. 24 is a schematic diagram illustrating an exemplary process for determining first feature points according to some embodiments of the present disclosure;

FIG. 25 is a schematic diagram illustrating an exemplary process for determining a second transformation relationship according to some embodiments of the present disclosure;

FIG. 26 is a schematic diagram illustrating an exemplary target portion according to some embodiments of the present disclosure;

FIG. 27 is a flowchart illustrating an exemplary process 2700 for determining updated positioning information of a robot according to some embodiments of the present disclosure;

FIG. 28 is a schematic diagram illustrating exemplary updated target postures according to some embodiments of the present disclosure;

FIG. 29 is a flowchart illustrating an exemplary process for adjusting an image capturing apparatus according to some embodiments of the present disclosure;

FIG. 30 is a schematic diagram illustrating an exemplary display interface according to some embodiments of the present disclosure;

FIG. 31 is a flowchart illustrating an exemplary process for adjusting an image capturing apparatus to a first target posture according to some embodiments of the present disclosure;

FIG. 32 is a schematic diagram illustrating an exemplary process for robot positioning according to some embodiments of the present disclosure;

FIG. 33 is a schematic diagram illustrating an exemplary process for robot positioning according to some embodiments of the present disclosure;

FIG. 34 is a flowchart illustrating an exemplary semi-automatic process for robot positioning according to some embodiments of the present disclosure;

FIG. 35 is a flowchart illustrating an exemplary process for robot positioning according to some embodiments of the present disclosure;

FIG. 36 is a schematic diagram illustrating an exemplary display interface according to some embodiments of the present disclosure; and

FIG. 37 is a schematic diagram illustrating an exemplary process for determining a first feature point according to some embodiments of the present disclosure.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously obtained from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if other words accomplish the same purpose.

As indicated in the present disclosure and in the claims, unless the context clearly suggests an exception, the words “one,” “a,” “a kind of,” and/or “the” do not refer specifically to the singular but may also include the plural. In general, the terms “including” and “comprising” suggest only the inclusion of clearly identified steps and elements, which do not constitute an exclusive list, and the method or device may also include other steps or elements.

The present disclosure uses flowcharts to illustrate the operations performed by the system according to some embodiments of the present disclosure. It should be understood that the operations described herein are not necessarily executed in a specific order. Instead, they may be executed in reverse order or simultaneously. Additionally, other operations may be added to these processes or certain steps may be removed.

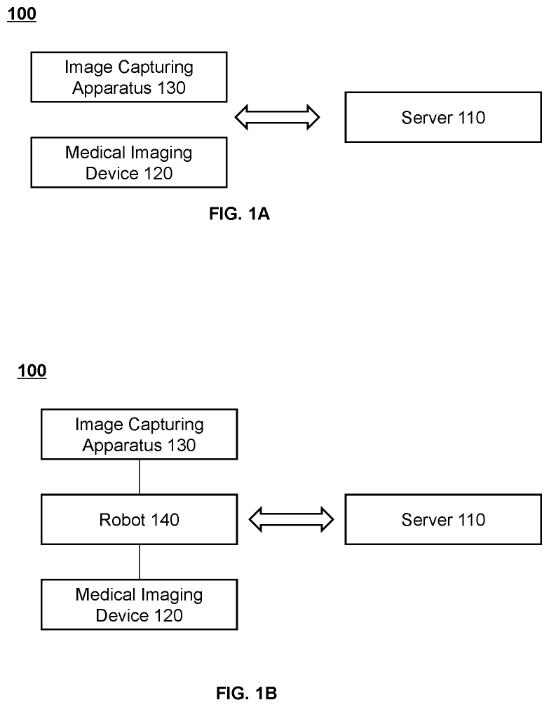

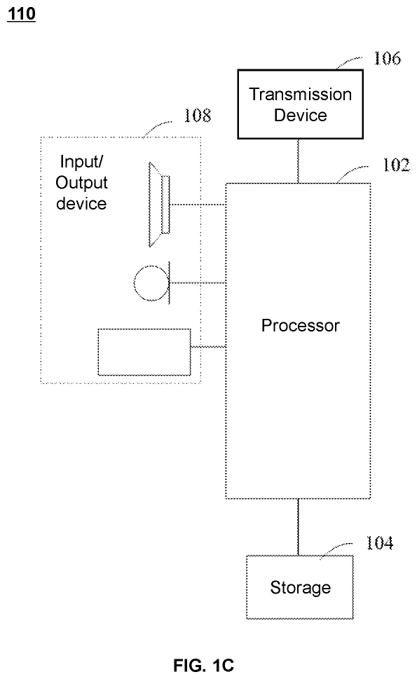

FIG. 1A is a schematic diagram illustrating an application scenario of an exemplary robotic control system 100 according to some embodiments of the present disclosure.

The robotic control system 100 may be used for positioning a robot and adjusting a posture of the robot. As shown in FIG. 1A, in some embodiments, the robotic control system 100 may include a server 110, a medical imaging device 120, and an image capturing apparatus 130. The plurality of components of the robotic control system 100 may be connected to each other via a network. For example, the server 110 and the medical imaging device 120 may be connected or in a communication through a network. As another example, the server 110 and the image capturing apparatus 130 may be connected or in a communication through a network. In some embodiments, connections between the plurality of components of the robotic control system 100 may be variable. For example, the medical imaging device 120 may be directly connected to the image capturing apparatus 130.

The server 110 may be configured to process data or information received from at least one component (e.g., the medical imaging device 120, the image capturing apparatus 130) of the robotic control system 100 or an external data source (e.g., a cloud data center). For example, the server 110 may obtain a first image captured by the image capturing apparatus 130 and a second image captured by the medical imaging device 120, and determine the positioning information of the robot based on the first image and the second image. As another example, the server 110 may capture a target image of a target object using the image capturing apparatus 130, and determine a first target posture of the image capturing apparatus 130 in a base coordinate system. In some embodiments, the server 110 may be a single server or a server group. The server group may be centralized or distributed (e.g., the server 110 may be a distributed system). In some embodiments, the server 110 may be local or remote. In some embodiments, the server 110 may be implemented on a cloud platform or provided virtually. Merely by way of example, the cloud platform may include a private cloud, a public cloud, a hybrid cloud, a community cloud, a distributed cloud, an internal cloud, a multi-tiered cloud, or any combination thereof.

In some embodiments, the server 110 may include one or more components. As shown in FIG. 1C, the server 110 may include one or more (only one shown in FIG. 1C) processors 102, storages 104, transmission devices 106, and input/output devices 108. It is understood by those skilled in the art that the structure shown in FIG. 1C is merely for purposes of illustration, and does not limit the structure of the server 110. For example, the server 110 may include more or fewer components than those shown in FIG. 1C, or may have a configuration different from that shown in FIG. 1C.

The processor 102 may process data or information obtained from other devices or components of the system. The processor 102 may execute program instructions based on the data, the information, and/or processing results, to perform one or more functions described in the present disclosure. In some embodiments, the processor 102 may include one or more sub-processing devices (e.g., a single-core processing device or a multi-core multi-processor device). Merely by way of example, the processor 102 may include a microprocessor unit (MPU), a central processing unit (CPU), an application-specific integrated circuit (ASIC), an application-specific instruction processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction set computer (RISC), or the like, or any combination thereof. In some embodiments, the processor 102 may be integrated or included in one or more other components (e.g., the medical imaging device 120, the image capturing apparatus 130, or other possible components) of the robotic control system 100.

The storage 104 may store data, instructions, and/or any other information. For example, the storage 104 may be configured to store a computer program such as a software program and module for an application, for example, a computer program corresponding to positioning methods and posture adjustment methods in the embodiment. The processor 102 may perform various functional applications and data processing by executing the computer program stored in the storage 104, thereby implementing the methods described above. The storage 104 may include high-speed random-access memory and may further include non-volatile memory, such as one or more magnetic storage devices, flash memory, or other non-volatile solid-state storage. In some embodiments, the storage 104 may also include a remote storage configured relative to the processor 102. The remote storage may be connected to a terminal via a network. Examples of the network may include the Internet, intranets, local area networks, mobile communication networks, or any combination thereof. In some embodiments, the storage 104 may be implemented on a cloud platform.

The communication device 106 may be configured to implement communication functions. For example, the communication device 106 may be configured to receive or transmit data via a network. In some embodiments, the communication device 106 may include a network interface controller (NIC) that can communicate with other network devices via a base station to communicate with the Internet. In some embodiments, the communication device 106 may be a radio frequency (RF) module for wireless communication with the Internet.

The input/output device 108 may be configured to input or output signals, data, or information. In some embodiments, the input/output device 108 may facilitate communication between a user and the robotic control system 100. Exemplary input devices may include a keyboard, a mouse, a touch screen, a microphone, or the like, or any combination thereof. Exemplary output devices may include a display device, a speaker, a printer, a projector, or the like, or any combination thereof. Exemplary display devices may include a liquid crystal display (LCD), a light emitting diode (LED) display, a flat panel display, a curved display, a television, a cathode ray tube (CRT), or the like, or any combination thereof.

In some embodiments, the server 110 may be disposed at any location (e.g., a room where the robot is located, a room used for placing the server 110, etc.), as long as the location ensures that the server 110 is in a normal communication with the medical imaging device 120 and the image capturing apparatus 130.

The medical imaging device 120 may be configured to scan the target object in a detection region or a scanning region to obtain imaging data of the target object. In some embodiments, the target object may include a biological and/or a non-biological object. For example, the target object may be an organic and/or inorganic substance with or without life.

In some embodiments, the medical imaging device 120 may be a non-invasive imaging device for diagnostic or research purposes. For example, the medical imaging device 120 may include a single-model scanner and/or a multi-model scanner. The single-model scanner may include, for example, an ultrasound scanner, an X-ray scanner, a computed tomography (CT) scanner, a magnetic resonance imaging (MRI) scanner, an ultrasound examiner, a positron emission tomography (PET) scanner, an optical coherence tomography (OCT) scanner, an ultrasound (US) scanner, an intravascular ultrasound (IVUS) scanner, a near-infrared spectroscopy (NIRS) scanner, a far-infrared (FIR) scanner, or the like, or any combination thereof. The multi-model scanner may include, for example, an X-ray imaging-magnetic resonance imaging (X-ray-MRI) scanner, a positron emission tomography-X-ray imaging (PET-X-ray) scanner, a single-photon emission computed tomography-magnetic resonance imaging (SPECT-MRI) scanner, a positron emission tomography-computed tomography (PET-CT) scanner, a digital subtraction angiography-magnetic resonance imaging (DSA-MRI) scanner, or the like, or any combination thereof. The scanners are merely for purposes of illustration, and do not limit the scope of the present disclosure. Merely by way of example, the medical imaging device 120 may include a CT scanner.

The image capturing apparatus 130 may be configured to capture image data (e.g., the first image, the target image) of the target object. Exemplary image capturing apparatus may include a camera, an optical sensor, a radar sensor, a structured light camera, or the like, or any combination thereof. For example, the image capturing apparatus 130 may include a device capable of capturing optical image data of the target object, such as, the camera (e.g., a depth camera, a stereo triangulation camera, a binocular camera, etc.), the optical sensor (e.g., a red-green-blue-depth (RGB-D) sensor, etc.), etc. As another example, the image capturing apparatus 130 may include a device capable of capturing point cloud data of the target object, such as, a laser imaging device (e.g., a time-of-flight (TOF) laser capture device, a point laser capture device, a line laser capture device, etc.), etc. The point cloud data may include a plurality of data points, wherein each of the plurality of data points may represent a physical point on a body surface of the target object, and one or more feature values (e.g., feature values related to a position and/or a composition) of the physical point may be used to describe the target object. The point cloud data may be used to reconstruct an image of the target object. As still another example, the image capturing apparatus 130 may include a device capable of obtaining location data and/or depth data of the target object, such as, a structured light camera, a TOF device, a light triangulation device, a stereo matching device, or the like, or any combination thereof. The location data and/or the depth data obtained by the image capturing apparatus 130 may be used to reconstruct the image of the target object.

In some embodiments, the image capturing apparatus 130 may be installed on the robot in a detachable or non-detachable connection manner. For example, the image capturing apparatus 130 may be detachably disposed to an end terminal of a robotic arm of the robot. In some embodiments, the image capturing apparatus 130 may be installed at a location outside the robot using a detachable or non-detachable connection manner. For example, the image capturing apparatus 130 may be disposed at a fixed location in the room where the robot is located.

In some embodiments, a corresponding relationship between the image capturing apparatus 130 and the robot may be determined based on a position of the image capturing apparatus 130, a position of the robot, and a calibration parameter (e.g., a size, a capturing angle) of the image capturing apparatus 130. For example, a mapping relationship (i.e., a first transformation relationship) between a first coordinate system corresponding to the image capturing apparatus 130 and a second coordinate system corresponding to the robot may be determined.

It should be noted that the descriptions are provided for the purposes of illustration, and are not intended to limit the scope of the present disclosure. For persons having ordinary skills in the art, various variations and modifications may be conducted under the teaching of the present disclosure. The features, structures, methods, and other features of the exemplary embodiments described in the present disclosure can be combined in various manners to obtain additional and/or alternative exemplary embodiments. For example, the image capturing apparatus 130 may include a plurality of image capturing apparatus.

In some embodiments, as shown in FIG. 1B, the robot control system 100 may also include a robot 140.

The robot 140 may perform a corresponding operation based on an instruction. For example, the robot 140 may perform a movement operation (e.g., translation, rotation, etc.) based on a movement instruction. Exemplary robots may include a surgical robot, a rehabilitation robot, a bio-robot, a remote rendering robot, a follow-along robot, a disinfection robot, or the like, or any combination thereof.

Merely by way of example, the robot 140 may include a multi-degree-of-freedom robotic arm. The multi-degree-of-freedom robotic arm may include a base joint where a base of the robotic arm is located and an end joint where a flange of the robotic arm is located. The flange of the robotic arm is fixedly connected with end tools, such as surgical tools (e.g., electrode needles, puncture needles, syringes, ablation needles, etc.).

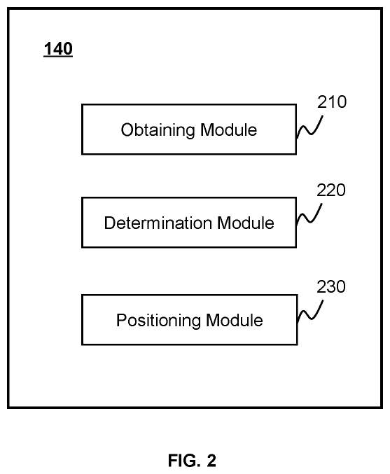

FIG. 2 is a block diagram illustrating an exemplary processor 102 according to some embodiments of the present disclosure. The processor 102 may include an obtaining module 210, a determination module 220, and a positioning module 230.

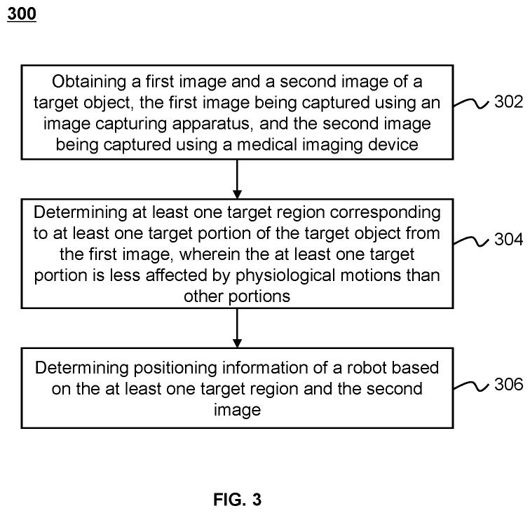

The obtaining module 210 may be configured to obtain a first image and a second image of a target object. The first image may be captured using an image capturing apparatus, and the second image may be captured using a medical imaging device. More descriptions regarding the obtaining the first image and the second image may be found in elsewhere in the present disclosure. See, e.g., operation 302 in FIG. 3 and relevant descriptions thereof.

The determination module 220 may be configured to determine at least one target region corresponding to at least one target portion of the target object from the first image. The at least one target portion may be less affected by physiological motions than other portions. More descriptions regarding the determination of the at least one target region may be found in elsewhere in the present disclosure. See, e.g., operation 304 in FIG. 3 and relevant descriptions thereof.

The positioning module 230 may be configured to determine positioning information of a robot based on the at least one target region and the second image. The positioning information refers to location information of the robot or a specific component (e.g., an end terminal of a robotic arm for mounting a surgical instrument) thereof. In some embodiments, the positioning module 230 may obtain a first transformation relationship between a first coordinate system corresponding to the image capturing apparatus and a second coordinate system corresponding to the robot. The positioning module 230 may further determine a second transformation relationship between the first coordinate system and a third coordinate system corresponding to the medical imaging device based on a registration relationship between the at least one target region and the second image. The positioning module 230 may determine the positioning information of the robot based on the first transformation relationship and the second transformation relationship. More descriptions regarding the determination of the positioning information of the robot may be found in elsewhere in the present disclosure. See, e.g., operation 306 in FIG. 3 and relevant descriptions thereof.

All or some of the modules of the robot control system described above may be implemented through a software, a hardware, or a combination thereof. These modules may be hardware components embedded in or separated from the processor of a computing device, or may be stored in a storage of a computing device in software form, so as to be retrieved by the processor to perform the operations corresponding to each module.

It should be noted that the descriptions of the robot control system and the modules thereof are provided for convenience of illustration, and are not intended to limit the scope of the present disclosure. It should be understood that those skilled in the art, having an understanding of the principles of the system, may arbitrarily combine the various modules or constitute subsystems connected to other modules without departing from the principles. For example, the obtaining module 210, the determination module 220, and the positioning module 230 disclosed in FIG. 2 may be different modules in the same system, or may be a single module that performs the functions of the modules mentioned above. As another example, modules of the robot control system may share a storage module, or each module may have an own storage module. Such modifications may not depart from the scope of the present disclosure.

FIG. 3 is a flowchart illustrating an exemplary process 300 for robot positioning according to some embodiments of the present disclosure. In some embodiments, the process 300 may be implemented by the robot control system 100. For example, the process 300 may be stored in a storage device (e.g., the storage 104) in the form of an instruction set (e.g., an application). In some embodiments, the processor 102 (e.g., the one or more modules as shown in FIG. 2) may execute the instruction set and direct one or more components of the robot control system 100 to perform the process 300.

Robots are widely used in the medical field. To accurately control operations of the robots, it is necessary to position the robots. A marker-based positioning technique is commonly used to position robots. Taking neurosurgery as an example, markers need to be implanted in the skull of a patient or attached to the head of the patient, and medical scans are performed on the patient with the markers. Furthermore, corresponding position information of the markers in an image space and a physical space may be determined, thereby positioning the robot based on a corresponding relationship between the image space and the physical space. However, the markers usually cause additional harm to the patient. In addition, once there is a relative displacement between the markers and the head of the patient in the preoperative images, the accuracy of the robot positioning is reduced, thereby affecting preoperative planning or surgical operations. Therefore, it is necessary to provide an effective system and method for robot positioning. In some embodiments, the robot may be positioned by performing the following operations in the process 300.

In 302, the processor 102 (e.g., the obtaining module 210) may obtain a first image and a second image of a target object. The first image may be obtained using an image capturing apparatus, and the second image may be obtained using a medical imaging device.

In some embodiments, the target object may include a biological object and/or a non-biological object. For example, the target object may be an organic and/or inorganic substance with or without life. As another example, the target object may include a specific part, organ, and/or tissue of a patient. Merely by way of example, in a scenario of neurosurgery, the target object may be the head or face of the patient.

The first image refers to an image obtained using the image capturing apparatus (e.g., the image capturing apparatus 130). The first image may include a three-dimensional (3D) image and/or a two-dimensional (2D) image. In some embodiments, the first image may include a depth image of the target object, which includes distance information from points on the surface of the target object to a reference point.

In some embodiments, the processor 102 may obtain image data of the target object from the image capturing apparatus (e.g., the image capturing apparatus 130), and determine the first image of the target object based on the image data. For example, when the image capturing apparatus is a camera, the processor 102 may obtain optical data of the target object from the camera, and determine the first image based on the optical data. As another example, when the image capturing apparatus is a laser imaging device, the processor 102 may obtain point cloud data of the target object from the laser imaging device, and determine the first image based on the point cloud data. As still another example, when the image capturing apparatus is a depth camera, the processor 102 may obtain depth data of the target object from the depth camera, and generate a depth image based on the depth data as the first image. In some embodiments, the processor 102 may directly obtain the first image from the image capturing apparatus or a storage device (e.g., the storage 104).

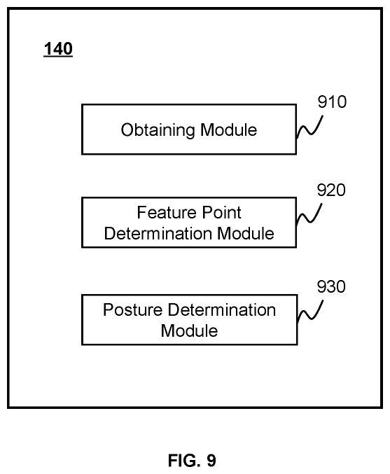

In some embodiments, before the first image of the target object is captured using the image capturing apparatus, a surgical position of the target object may be determined based on preoperative planning. The target object may be fixed, and a posture of the image capturing apparatus may be adjusted such that the image capturing apparatus captures the target object from a target shooting angle and/or a target shooting height. For example, the posture of the image capturing apparatus may be adjusted such that the face of a patient is completely within a field of view of the image capturing apparatus, and the image capturing apparatus is aligned vertically with the face of the patient for imaging. More descriptions regarding the adjustment of the posture of the image capturing apparatus may be found in elsewhere in the present disclosure. See, e.g., FIGS. 9-17 and relevant descriptions thereof.

The second image refers to a medical image captured using a medical imaging device (e.g., the medical imaging device 120). Merely by way of example, the medical imaging device may be a CT device. Correspondingly, the processor 102 may obtain CT image data of the target object using the CT device, and reconstruct a CT image based on the CT image data. The processor 102 may further obtain the second image by performing a 3D reconstruction on the CT image.

In some embodiments, the processor 102 may directly obtain the second image of the target object from the medical imaging device (e.g., the medical imaging device 120). Alternatively, the processor 102 may obtain the second image of the target object from a storage device (e.g., the storage 104) that stores the second image of the target object.

In some embodiments, the processor 102 may first obtain a first initial image and/or a second initial image, wherein the first initial image is captured using the image capturing apparatus, and the second initial image is captured using the medical imaging device. The processor 102 may generate the first image and/or the second image by processing the first initial image and/or the second initial image. Merely by way of example, the processor 102 may obtain a full-body depth image and a full-body CT image of a patient. The processor 102 may obtain the first image by segmenting a portion corresponding to the face of the patient from the full-body depth image. The processor 102 may obtain a 3D reconstructed image by performing a 3D reconstruction on the full-body CT image, and obtain the second image by segmenting a portion corresponding to the face of the patient from the 3D reconstructed image.

In some embodiments, after the first image and the second image of the target object are obtained, the processor 102 may perform a preprocessing operation (e.g., target region segmentation, dimension adjustment, image resampling, image normalization, etc.) on the first image and the second image. The processor 102 may further perform other operations of the process 300 on the preprocessed first image and the preprocessed second image. For purposes of illustration, the first image and the second image are taken as examples for describing the execution process of the process 300.

In 304, the processor 102 (e.g., the determination module 220) may determine at least one target region corresponding to at least one target portion of the target object from the first image. The at least one target region determined from the first image is also referred to as at least one first target region.

In some embodiments, the at least one target portion may be less affected by physiological motions than other portions, such target portion is also referred to as a first target portion. The physiological motions may include blinking, respiratory motions, cardiac motions, etc. Merely by way of example, the at least one target portion may be a static facial region. The static facial region refers to a region that is less affected by changes in facial expressions, such as, a region near a facial bone structure. In some embodiments, the processor 102 may capture shape data of human faces under different facial expressions, obtain a region that is less affected by the changes in facial expressions by performing a statistical analysis on the shape data, and determine the region as the static facial region. In some embodiments, the processor 102 may determine the static facial region using physiological structure information. For example, the processor 102 may determine a region close to the facial bone structure as the static facial region. Exemplary static facial regions may include a forehead region, a nasal bridge region, etc.

A target region refers to a region corresponding to a target portion of the target object from the first image. In some embodiments, the processor 102 may determine the at least one target region corresponding to the at least one target portion of the target object from the first image using an image recognition technique (e.g., a 3D image recognition model). Merely by way of example, the processor 102 may input the first image into the 3D image recognition model, and the 3D image recognition model may segment the at least one target region from the first image. The 3D image recognition model may be obtained by training, based on a plurality of training samples, an initial model. Each of the plurality of training samples may include a sample first image of a sample object and a corresponding sample target region, wherein the sample first image is determined as a training input, and the corresponding sample target region is determined as a training label. In some embodiments, the processor 102 (or other processing devices) may iteratively update the initial model based on the plurality of training samples until a specific condition is met (e.g., a loss function is less than a certain threshold, a certain count of training iterations is performed).

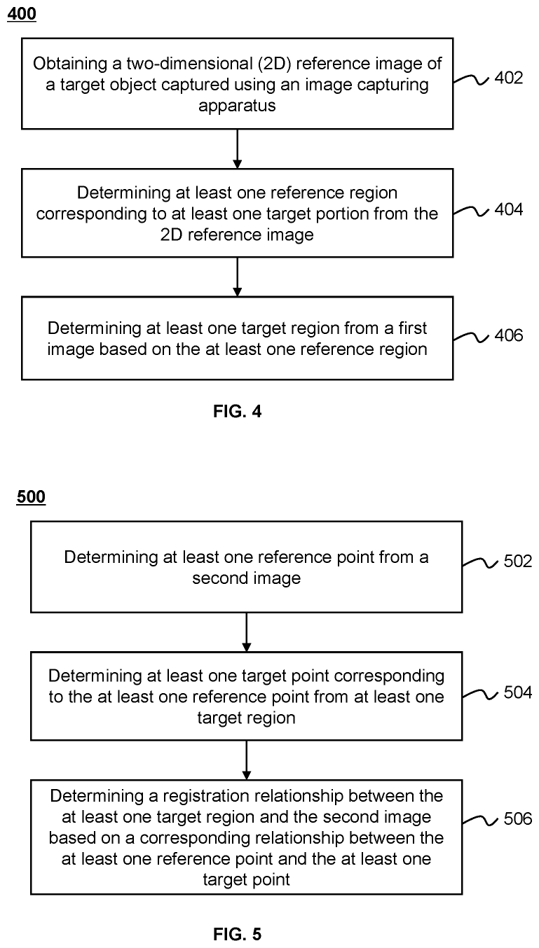

In some embodiments, the first image may be a 3D image (e.g., a 3D depth image). The processor 102 may obtain a 2D reference image of the target object captured using the image capturing apparatus. The processor 102 may determine at least one reference region corresponding to the at least one target portion based on the 2D reference image. Further, the processor 102 may determine the at least one target region from the first image based on the at least one reference region. More descriptions regarding the determination of the at least one target region may be found in elsewhere in the present disclosure. See, e.g., FIG. 4 and relevant descriptions thereof. When the first image is a 3D depth image, the at least one target region determined from the first image is also referred as first point cloud data.

In 306, the processor 102 (e.g., the positioning module 230) may determine positioning information of a robot based on the at least one target region and the second image.

The positioning information of the robot refers to location information of the robot or a specific component (e.g., an end terminal of a robotic arm for mounting a surgical instrument) thereof. For convenience of illustration, the positioning information of the specific component of the robot is referred to as the positioning information of the robot later in the present disclosure. In some embodiments, the positioning information of the robot may include a positional relationship between the robot and a reference object (e.g., the target object, a reference object determined by a user and/or the system), a transformation relationship between a coordinate system corresponding to the robot (i.e., a second coordinate system) and other coordinate systems (e.g., a first coordinate system, a third coordinate system), etc. For example, the positioning information may include a positional relationship between coordinates of the robot and coordinates of the target object in a same coordinate system.

In some embodiments, the processor 102 may obtain a first transformation relationship between the first coordinate system corresponding to the image capturing apparatus and the second coordinate system corresponding to the robot. The processor 102 may further determine a second transformation relationship between the first coordinate system and the third coordinate system corresponding to the medical imaging device based on a registration relationship between the at least one target region and the second image. The processor 102 may then determine the positioning information of the robot based on the first transformation relationship and the second transformation relationship. For example, the processor 102 may determine a third transformation relationship between the second coordinate system corresponding to the robot and the third coordinate system corresponding to the medical imaging device based on the first transformation relationship and the second transformation relationship.

The first coordinate system corresponding to the image capturing apparatus refers to a coordinate system established based on the image capturing apparatus, for example, a 3D coordinate system established with a geometric center of the image capturing apparatus as an origin. The second coordinate system corresponding to the robot refers to a coordinate system established based on the robot. For example, the second coordinate system may be a coordinate system of the end terminal of the robotic arm, a coordinate system of a tool of the robot, etc. The third coordinate system corresponding to the medical imaging device refers to a coordinate system established based on the medical imaging device, for example, a 3D coordinate system established with a rotation center of a gantry of the medical imaging device as an origin. As used in the present disclosure, a transformation relationship between two coordinate systems may represent a mapping relationship between positions in the two coordinate systems. For example, the transformation relationship may be represented as a transformation matrix that can transform a first coordinate of a point in one coordinate system to a corresponding second coordinate in another coordinate system. In some embodiments, a transformation relationship between a coordinate system corresponding to a first object and a coordinate system corresponding to a second object may also be referred to as a relative positional relationship or a position mapping relationship between the first object and the second object. For example, the first transformation relationship may also be referred to as a relative positional relationship or a position mapping relationship between the image capturing apparatus and the robot. In some embodiments, the transformation relationship may further include a registration error between two coordinate systems.

In some embodiments, the processor 102 may determine the first transformation relationship using a preset calibration technique (e.g., a hand-eye calibration algorithm). For example, the processor 102 may construct an intermediate reference object, and determine the first transformation relationship based on a first coordinate of the intermediate reference object in the first coordinate system (or a relative positional relationship between the intermediate reference object and the image capturing apparatus) and a second coordinate of the intermediate reference object in the second coordinate system (or a relative positional relationship between the intermediate reference object and the robot). The image capturing apparatus may be installed on the robot, for example, at the end terminal of a manipulator arm of the robot. Alternatively, the image capturing apparatus may be disposed at any fixed location in a room where the robot is located, and the processor 102 may determine a mapping relationship between a position of the robot and a position of the image capturing apparatus based on the position of the image capturing apparatus, the position of the robot, and a position of the intermediate reference object.

In some embodiments, the processor 102 may determine the second transformation relationship between the first coordinate system corresponding to the image capturing apparatus and the third coordinate system corresponding to the medical imaging device based on the registration relationship between at least one target region and the second image. The registration relationship may reflect a corresponding relationship and/or a coordinate transformation relationship between points in the at least one target region and points in the second image. Since the at least one target region and the second image correspond to the same target object, there may be the corresponding relationship between the points in the at least one target region and the points in the second image, so the registration relationship between the at least one target region and the second image can be determined through a registration technique. More descriptions regarding the determination of the registration relationship between the at least one target region and the second image may be found in elsewhere in the present disclosure. See, e.g., FIG. 5 and relevant descriptions thereof.

According to some embodiments of the present disclosure, the first image and the second image of the target object may be obtained, the at least one target region corresponding to the at least one target portion of the target object may be determined from the first image, and the positioning information of the robot may be determined based on the at least one target region and the second image. The transformation relationships between the coordinate systems of the robot, the image capturing apparatus, and the medical imaging device may be determined through the at least one target region and the second image (i.e., a medical image), and no additional markers need to be attached or disposed on the target object, thereby avoiding additional harm to the target object. Using the at least one target region and the second image for registration, instead of directly using the first image and the second image for registration, the influence of the physiological motions on a registration result can be reduced, thereby improving the accuracy of the registration result. In addition, the robot can be positioned based on the medical image, which can improve the accuracy of the robot positioning, thereby improving the accuracy of preoperative planning or surgical operations.

It should be noted that the above descriptions of the process 300 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the process 300 may be accomplished with one or more additional operations not described, and/or without one or more of the operations discussed. For example, after the positioning information of the robot is determined, the processor 102 may verify the positioning information to ensure the accuracy of the robot positioning. As another example, the processor 102 may control the robot to perform a surgical plan. For instance, the processor 102 may control the robot to move to a target location and perform surgical operations based on the positioning information and the surgical plan.

FIG. 4 is a flowchart illustrating an exemplary process 400 for determining at least one target region according to some embodiments of the present disclosure. In some embodiments, the process 400 may be implemented by the robot control system 100. For example, the process 400 may be stored in a storage device (e.g., the storage 104) in the form of an instruction set (e.g., an application). In some embodiments, the processor 102 (e.g., the one or more modules as shown in FIG. 2) may execute the instruction set and direct one or more components of the robot control system 100 to perform the process 400. In some embodiments, the at least one target region described in operation 304 of FIG. 3 may be determined according to the process 400.

In 402, the processor 102 (e.g., the determination module 220) may obtain a 2D reference image of a target object captured using an image capturing apparatus (also referred to as a first 2D reference image).

In some embodiments, the first image described in FIG. 3 may be a 3D image, such as a depth image. The processor 102 may directly obtain the 2D reference image of the target object from the image capturing apparatus or a storage device. For example, the image capturing apparatus may be a depth camera or a laser capturing device, which can simultaneously capture the 2D reference image and the depth image of the target object. The 2D reference image may be an RGB image. In some embodiments, both the 2D reference image and the first image are captured by the image capturing apparatus with a first target posture. In some embodiments, the processor 102 may generate the 2D reference image of the target object based on the first image. For example, the processor 102 may transform the 3D first image into the 2D reference image using an image transformation algorithm.

In 404, the processor 102 (e.g., the determination module 220) may determine at least one reference region corresponding to at least one target portion from the 2D reference image.

A reference region refers to a region in the 2D reference image corresponding to a target portion. For example, FIG. 8 is a schematic diagram illustrating an exemplary 2D reference image according to some embodiments of the present disclosure. As shown in FIG. 8, shaded regions may be reference regions corresponding to facial static regions, e.g., the forehead, the bridge of the nose, etc.

In some embodiments, the processor 102 may determine at least one feature point associated with the at least one target portion from the 2D reference image (also referred to as at least one first feature point). For example, the processor 102 may determine the at least one feature point associated with the at least one target portion from the 2D reference image based on a preset feature point extraction algorithm. Exemplary feature point extraction algorithms may include a scale-invariant feature transform (SIFT) algorithm, a speeded-up robust features (SURF) algorithm, a histogram of oriented gradient (HOG) algorithm, a difference of gaussian (DOG) algorithm, a feature point extraction algorithm based on a machine learning model, or the like, or any combination thereof.

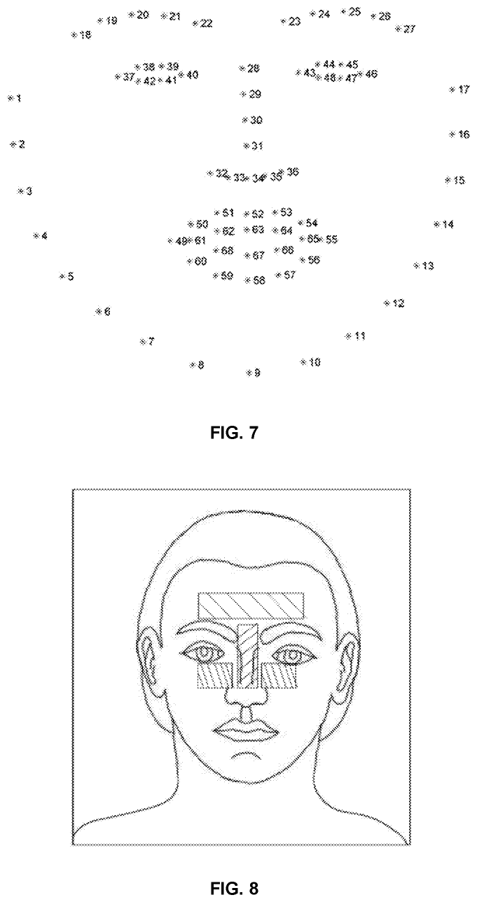

For example, the processor 102 may perform feature point extraction on the 2D reference image using an image feature point extraction model (e.g., a pre-trained neural network model). The processor 102 may input the 2D reference image into the image feature point extraction model, and the image feature point extraction model may output the at least one feature point associated with the at least one target portion in the 2D reference image. Merely by way of example, the 2D reference image may be a 2D facial image. The at least one feature point in the 2D facial image associated with the at least one target portion may be obtained by inputting the 2D facial image into the image feature point extraction model (e.g., a face feature point detection model). As shown in FIG. 7, facial feature points identified from the 2D facial image may include feature points of the eyes, the mouth, the eyebrows, the nose, etc.

Further, the processor 102 may determine the at least one reference region corresponding to the at least one target portion based on the at least one feature point. For example, a region of the eyes may be determined based on the feature points of the eyes. In some embodiments, each of the at least one feature point may have a fixed sequence number, and the at least one reference region may be determined from the 2D reference image based on the fixed sequence number of the feature point. For example, referring to FIG. 7 again, feature points with sequence numbers of 37 to 42 in the facial feature points are feature points corresponding to a right eye, and a right eye region may be determined based on the feature points.

In some embodiments, the processor 102 may determine the at least one reference region corresponding to the at least one target portion from the 2D reference image through an image identification technique (e.g., a 2D image identification model). Merely by way of example, the processor 102 may input the 2D reference image into the 2D image identification model, and the 2D image identification model may segment the at least one reference region from the 2D reference image. The 2D image identification model may be obtained by training based on training samples. Each of the training samples may include a sample 2D reference image of a sample object and a corresponding sample reference region. The sample 2D reference image may be a training input, and the corresponding sample reference region may be a training label. In some embodiments, the processor 102 (or other processing devices) may iteratively update an initial model based on the training samples until a specific condition is met (e.g., a loss function is less than a certain threshold, a count of training iterations reaches a certain count, etc.).

In some embodiments, the at least one feature point includes multiple feature points, the at least one reference region may be a region covering each feature point in the 2D reference image or a region determined by connecting the feature points. For example, as shown in FIG. 26, a region 2602 is a region covering each feature point in the 2D reference image, and a region 2604 is a region determined by connecting the feature points. In some embodiments, both the region 2602 and the region 2604 are determined as the at least one reference region.

In 406, the processor 102 (e.g., the determination module 220) may determine at least one target region from the first image based on the at least one reference region.

In some embodiments, the processor 102 may determine the at least one target region corresponding to the at least one reference region based on a mapping relationship between the 2D reference image and the first image. The mapping relationship between the 2D reference image and the first image may be determined based on parameter(s) of the image capturing apparatus.

In some embodiments, the processor 102 may perform identification on the first feature points from the first 2D reference image captured by the image capturing apparatus with the first target posture. If the identification of one or more of the first feature points fails, the processor 102 may present the first 2D reference image for guiding a user to label the first feature points on the first 2D reference image. The processor 102 may determine at least one first target region corresponding to the at least one target portion of the target object from the first image based on the first feature points labelled by the user, and determine positioning information of a robot based on the at least one first target region and the second image. For example, the processor 102 may present the first 2D reference image on a display interface to prompt the user to label the first feature points on the first 2D reference image. As another example, the processor 102 may present a guidance image and the first 2D reference image on the display interface to prompt the user to label the first feature points on the first 2D reference image.

In some embodiments, the first 2D reference image captured by the image capturing apparatus may be displayed for the user to observe. To facilitate better viewing of the first 2D reference image, after the first 2D reference image is captured, the first 2D reference image may be automatically rotated according to a transformation relationship between the target object (e.g., the patient's face) and the base coordinate system, so that the first 2D reference image faces the user, thereby facilitating the user's observation of an acquisition process of facial point cloud data corresponding to the first image. If the automatic image rotation fails, a manual rotation button may be provided, allowing the user to manually rotate the first 2D reference image to a desired orientation. For example, the display interface may provide a rotation button, and the user may rotate the first 2D reference image displayed on the display interface through gestures or button presses.

To enable the user to mark the first feature points more effectively, the guidance image may be displayed on the display interface to prompt the user on how to label the physiological feature points. The guidance image may include first position information of the first feature points. The user may determine second position information to be labelled on the first 2D reference image based on the first position information of the first feature points in the guidance image. For example, the guidance image may be an image shown in FIG. 23. During the user's labelling of each first feature point, the first feature point selected by the user on the first 2D reference image and its corresponding position may be acquired. When the second position information for each first feature point is acquired, labelling may be determined to be complete.

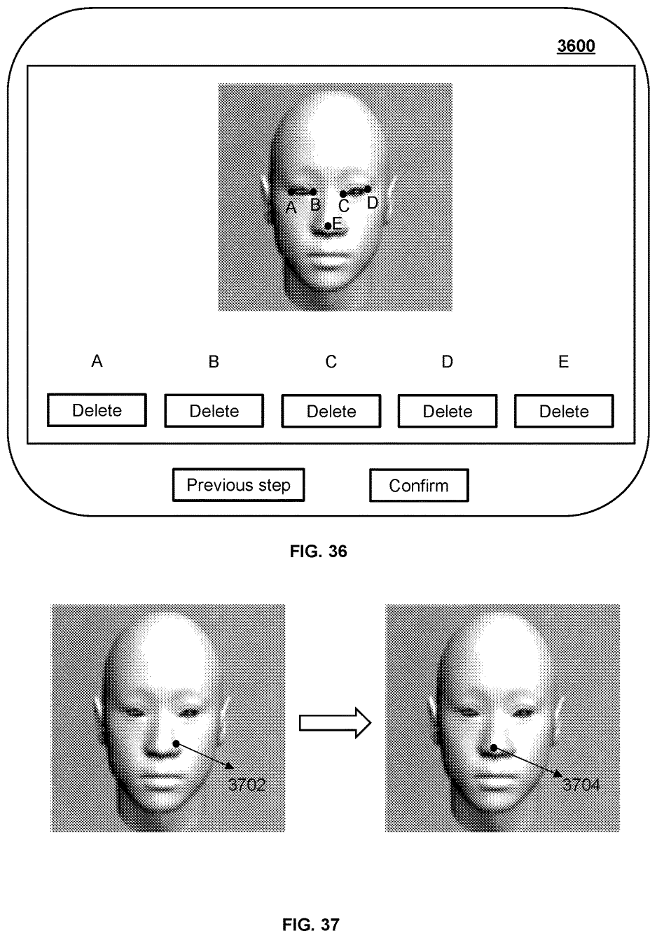

Merely by way of example, referring to FIG. 36, a display interface 3600 may present candidate first feature points A, B, C, D, and E. If the user determines that the candidate first feature points A, B, C, D, and E are accurate, he/she may reserve those feature points as the first feature points. For instance, when the user clicks a valid position in the image, the five buttons in the display interface 3600 may become activatable, for example, the buttons may be displayed in green. When the buttons are activatable, the user may click one of the buttons, and a candidate feature point corresponding to the button clicked by the user may be a first feature point corresponding to the valid position previously clicked by the user. The display interface 3600 may generate a label for the first feature point corresponding to the button clicked by the user at that valid position. The valid position may be a position within the facial region. When the user clicks a position outside the facial region, the click may be considered invalid, and the five buttons in the display interface 3600 may become inactivatable, for example, the five buttons in the interface may be grayed out. When the buttons are inactivatable, the user cannot label the clicked position via the buttons, thereby preventing the user from labelling invalid positions. As still another example, after a first feature point corresponding to one button on the display interface 3600 has been labelled by the user, the button may remain inactivatable during subsequent labelling processes to prevent duplicate labelling. When the user finds a mis-marked point, an undo command may be triggered, thereby deactivating the inactivatable state of the button and discarding the labelling position previously associated with the button, allowing the user to re-mark the first feature point corresponding to the button.

In some embodiments, the display interface 3600 may also include a “Previous Step” button, where the previous step button is used to return to a previous process of facial registration, i.e., returning to the process of positioning the image capturing apparatus. When the user deems the first 2D reference image in the display interface 3600 unsatisfactory, the user may click the previous step button, thereby readjusting the position of the image capturing apparatus and recapturing a satisfactory first 2D reference image. In some embodiments, during the labelling of the first feature points, the position of the image capturing apparatus may be in a locked state, meaning the robotic arm's position is immovable and the image capturing apparatus's position is immovable. Generally, when the positioning process returns from labelling the first feature points to adjusting the position of the image capturing apparatus, the position of the image capturing apparatus is usually not far from its desired position, and the user may rotate the image capturing apparatus to reach the desired position. Therefore, after the user clicks the previous step button, only the locked state of the image capturing apparatus may be released, while the robotic arm may remain locked, allowing the user to adjust the position of the image capturing apparatus by rotating the image capturing apparatus. In some embodiments, the locked states of both the robotic arm and the image capturing apparatus may also be released simultaneously, allowing the user to drag the robotic arm and rotate the image capturing apparatus to adjust the first 2D reference image.

The display interface 3600 may also include a “Confirm” button. When not all five first feature points are labelled in the first 2D reference image, the confirm button may be in an inactivatable state, thereby prompting the user to continue labelling the first feature points. When all first feature points in the first 2D reference image are labelled, the confirm button may switch to an activatable state. When the user clicks the confirm button, the second position information of the first feature points labelled by the user in the first 2D reference image may be identified, and the facial point cloud data may be extracted based on the second position information.

As another example, as shown in FIG. 37, a candidate first feature point 3702 may be automatically determined in a first 2D reference image by performing identification on the first 2D reference image. If the user determines that the candidate first feature point 3702 is inaccurate, he/she may move the candidate first feature point 3702 to determine a first feature point 3704. As another example, the user may confirm the candidate first feature point 3702 as the first feature point 3704. As still another example, the user may delete the candidate first feature point 3702, and label the first feature point 3704.

According to some embodiments of the present disclosure, the at least one target region corresponding to the at least one target portion of the target object may be determined based on the at least one reference region corresponding to the at least one target portion determined from the 2D reference image. Compared to directly determining the at least one target region from the first image, by determining the at least one target region based on the 2D reference image, an impact of a depth parameter in the first image on the determination of the at least one target region can be reduced, thereby improving the accuracy of the target region determination. In addition, during the determination process, only one 2D reference image and one first image are processed, which can reduce the data volume during the determination process, thereby saving time and resources for data processing.

It should be noted that the above descriptions of the process 400 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the process 400 may be accomplished with one or more additional operations not described, and/or without one or more of the operations discussed. For example, the at least one target region may be determined using a plurality of 2D reference images and the first image. As another example, at least one corresponding feature point may be determined from the first image based on the at least one feature point, and then the at least one target region may be determined based on the at least one corresponding feature point.

FIG. 5 is a flowchart illustrating an exemplary process 500 for determining a registration relationship according to some embodiments of the present disclosure. In some embodiments, the process 500 may be implemented by the robot control system 100. For example, the process 500 may be stored in a storage device (e.g., the storage 104) in the form of an instruction set (e.g., an application). In some embodiments, the processor 102 (e.g., the one or more modules as shown in FIG. 2) may execute the instruction set and direct one or more components of the robot control system 100 to perform the process 500. In some embodiments, the registration relationship described in operation 306 of FIG. 3 may be determined according to the process 500.

In some embodiments, a registration relationship between at least one target region and a second image may be determined through a registration technique. Exemplary registration techniques may include a global registration technique, a local registration technique, etc. The global registration technique may be used for registration based on a corresponding relationship between a target plane in the at least one target region and a reference plane in the second image. The local registration may be used for registration based on a corresponding relationship between target points in the at least one target region and reference points in the second image. Merely by way of example, the registration relationship between the at least one target region and the second image may be determined through the global registration technique and/or the local registration technique.

In 502, the processor 102 (e.g., the positioning module 230) may determine at least one reference point from the second image.

In some embodiments, the at least one reference point may form at least one reference point set, and each of the at least one reference point set may include at least three reference points located on a same plane. In other words, each reference point set (e.g., the at least three reference points) may determine a reference plane from the second image. Operation 502 is used to determine at least one reference plane from the second image. In some embodiments, a count of reference points in each reference point set may be determined based on actual condition(s). For example, each reference point set may include at least four reference points located on a same plane. It should be noted that three points are sufficient to define a plane, but using a reference point set including at least four reference points can improve the accuracy of the determination of a positional relationship of reference points in the reference plane in a medical image, thereby improving the accuracy of the determination of the registration relationship.

In some embodiments, the processor 102 may randomly determine the at least one reference point from the second image, and determine position information of the at least one reference point in a third coordinate system corresponding to a medical imaging device. For example, the processor 102 may determine four reference points located in a same plane from the second image through a random sampling algorithm, and determine corresponding coordinates of the four reference points in the third coordinate system corresponding to the medical imaging device. The four reference points may form a reference point set.

In 504, the processor 102 (e.g., the positioning module 230) may determine at least one target point corresponding to the at least one reference point from the at least one target region.

In some embodiments, the processor 102 may determine the at least one target point corresponding to the at least one reference point in the at least one target region through the global registration technique.

In some embodiments, for each reference point set in the at least one reference point set, the processor 102 may determine positional relationships between reference point pairs among the reference point set. The reference point pairs may include adjacent reference points or non-adjacent reference points. A positional relationship between one reference point pair may include a distance between the reference points in the reference point pair, a relative direction between the reference points in the reference point pair, etc. In some embodiments, the positional relationships between the reference point pairs in a reference point set may be represented through vector information. For example, the processor 102 may determine a distance and a direction between each two reference points in the reference point set based on coordinates of the at least three reference points in the reference point set, and represent the distance and the direction through vector information. In other words, the processor 102 may determine vector information of the reference point set based on the distance and the direction between each two reference points among the reference point set.

In some embodiments, for each reference point set in the at least one reference point set, the processor 102 may determine a target point set corresponding to the reference point set from the at least one target region based on the positional relationships between the reference point pairs in the reference point set. Each target point set may include at least three target points located in a same plane. In other words, the processor 102 may determine a target plane from the at least one target region based on each target point set (e.g., the at least three target points). In some embodiments, the processor 102 may determine the target point set corresponding to the reference point set from the at least one target region based on the vector information of the reference point set. For example, the processor 102 may determine a plurality of candidate target point sets from the at least one target region. A count of candidate target points in each candidate target point set may be the same as a count of reference points in the reference point set. The processor 102 may further determine a candidate target point set that is most similar to the reference point set as the target point set. The most similar to the reference point set refers to that a difference between vector information of a candidate target point set and the vector information of the reference point set is minimum.

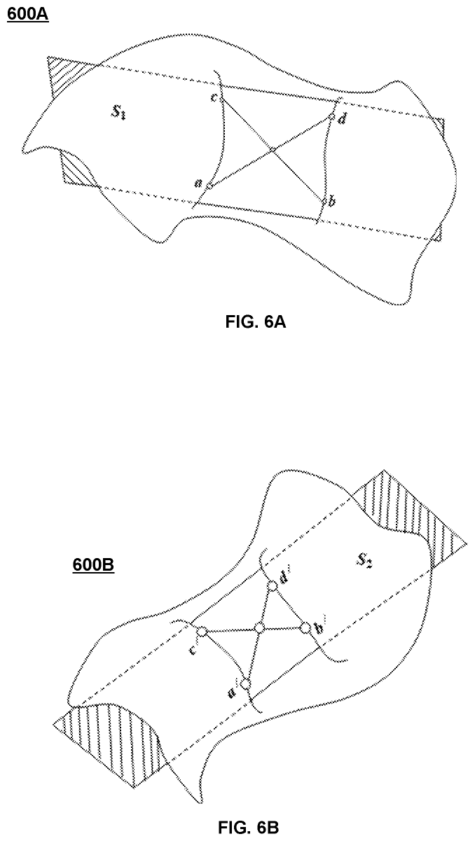

Merely by way of example, for each reference point set in the at least one reference point set, the processor 102 may determine a first distance between each two reference points among the reference point set based on the vector information of the reference point set, and determining a second distance between each two candidate target points among each of the plurality of candidate target point sets in the at least one target region. The processor 102 may determine a deviation between each first distance and the corresponding second distance. Further, the processor 102 may determine the target point set corresponding to the reference point set from the plurality of candidate target point sets based on the deviation between each first distance and the corresponding second distance. Referring to FIGS. 6A and 6B, FIG. 6A is a schematic diagram illustrating an exemplary reference point set 600A in a second image according to some embodiments of the present disclosure. As shown in FIG. 6A, the reference point set 600A in the second image may include four points (e.g., reference points) a, b, c, and d, and the four points a, b, c, and d may form a plane S1. The processor 102 may determine distances between point pairs a-b, a-c, a-d, b-c, b-d, and c-d as first distances based on position information (e.g., vector information) of the four points a, b, c, and d. FIG. 6B is a schematic diagram illustrating an exemplary candidate target point set 600B in at least one target region according to some embodiments of the present disclosure. As shown in FIG. 6B, the candidate target point set 600B may include four points (e.g., candidate target points) a′, b′, c′, and d′, and the four points a′, b′, c′, and d′ may form a plane S2. The processor 102 may determine distances between point pairs a′-b′, a′-c′, a′-d′, b′-c′, b′-d′, and c′-d′ as second distances based on position information of the four points a′, b′, c′, and d′.

For each of the first distances, the processor 102 may determine a deviation between the first distance and the corresponding second distance. The deviation may include a difference between the first distance and the second distance or a ratio of the first distance to the second distance. For example, the processor 102 may determine distance differences between point pairs a-b and a′-b′, a-c and a′-c′, . . . , c-d and c′-d′, respectively. As another example, the processor 102 may determine distance ratios of point pairs a-b to a′-b′, a-c to a′-c′, . . . , c-d to c′-d′, respectively. Further, the processor 102 may determine a difference between the candidate target point set and the reference point set based on the deviations between the first distances and the second distances. For example, the processor 102 may determine a sum of the distance differences, and determine the sum as the difference between the candidate target point set and the reference point set. As anther example, the processor 102 may determine an average of the distance differences, and determine the average as the difference between the candidate target point set and the reference point set. In some embodiments, the at least one target region may include a plurality of candidate target point sets. The processor 102 may determine a difference between each of the plurality of candidate target point sets and the reference point set. In some embodiments, the processor 102 may determine a candidate target point set with the minimum difference as the target point set.

The target point set may be determined based on the distance between each reference point pair in the reference point set and the distance between each candidate target point pair in the candidate target point set. Since the position information of each reference point and each candidate target point is known, the determination of the distances can be simple, thereby improving the efficiency of the determination of the target point set corresponding to the reference point set.

In some embodiments, the processor 102 may determine a target point corresponding to each reference point in the reference point set based on the target point set. For example, the processor 102 may determine, based on the target point set, a target point corresponding to each reference point through a positional corresponding relationship between each target point in the target point set and each reference point in the reference point set.

By using the global registration technique, the processor 102 may determine the target point set corresponding to the reference point set in the second image from the at least one target region, thereby determining the target points corresponding to the reference points. The implementation of the global registration technique is simple, which can improve the efficiency of the target point determination, and ensure the accuracy of the target point selection.

In some embodiments, the processor 102 may determine the target points corresponding to the reference points based on the local registration technique, thereby registering the reference points with the target points. For example, the processor 102 may determine the target points corresponding to the reference points through an iterative closest point (ICP) algorithm.

Merely by way of example, the processor 102 may obtain position information (e.g., coordinates, depth information, etc.) for each candidate target point in the at least one target region. The processor 102 may determine the target point corresponding to each reference point based on the position information of each candidate target point, the position information of each reference point, and a preset iterative closest point algorithm. For example, for each reference point, the processor 102 may determine, based on the iterative closest point algorithm, a target point with a closest distance (e.g., a Euclidean distance) to the reference point according to the position information of the reference point in the second image and the position information of each candidate target point in the at least one target region. For instance, the processor 102 may determine a reference point in the second image, search for a closest candidate target point in the at least one target region, and determine the closest candidate target point as the corresponding target point of the reference point. The processor 102 may determine, based on the reference points and the corresponding target points, a transformation matrix (e.g., a rotation matrix and/or a translation matrix) between the at least one target region and the second image. The processor 102 may transform the at least one target region based on the transformation matrix, and determine new target points corresponding to the reference points from at least one transformed target region. The above process may be iterated until a specific condition is met. For example, the specific condition may include that distances between the reference points and corresponding newest target points are less than a preset threshold, a count of iterations reaches a preset threshold, or differences between distances from the reference points to the corresponding newest target points and distances from the reference points to the corresponding previous target points are less than a preset threshold. The processor 102 may determine the registration relationship based on a corresponding relationship (e.g., the transformation matrix) between the reference points and target points in the last iteration.

In some embodiments, the processor 102 may also determine an initial registration relationship between the at least one target region and the second image based on the global registration technique as described above. Further, the processor 102 may determine the registration relationship by adjusting the initial registration relationship using the local registration technique (e.g., the iterative closest point algorithm).

Merely by way of example, the processor 102 may determine the initial registration relationship (i.e., an initial corresponding relationship (e.g., an initial transformation matrix)) between the target points in the at least one target region and the reference points in the second image based on the global registration technique. For each target point, the processor 102 may determine or adjust an initial reference point corresponding to each target point based on the preset iterative closest point algorithm. For example, for each target point, the processor 102 may determine a reference point with a closest distance (e.g., a Euclidean distance) to the target point from the reference points. If the reference point is different from the initial reference point in the initial registration relationship, the initial registration relationship may be updated, and the reference point with the closest distance to the target point may be determined as the corresponding new reference point. The processor 102 may determine, based on the target points and the corresponding new reference points, a transformation matrix (e.g., a rotation matrix and/or a translation matrix) between the at least one target region and the second image. The processor 102 may transform the at least one target region based on the transformation matrix, and determine new reference points from the second image based on transformed target points. The above process may be iterated until a specific condition is met. For example, the specific condition may include that distances between the transformed target points and the new reference points are less than a preset threshold, a count of iterations reaches a preset threshold, or differences between distances from the transformed target points to the new reference points and distances from the last reference points to the last target points are less than a preset threshold. The processor 102 may determine the registration relationship by adjusting the initial registration relationship between the at least one target region and the second image based on a corresponding relationship (e.g., the transformation matrix) between target points and reference points in the last iteration.

According to some embodiments of the present disclosure, the target points can be determined from the at least one target region based on the reference points in the second image, so that the registration relationship is determined based on the position information of the reference points and the target points. By registering with the reference points and the target points, data computation and processing time can be reduced, thereby simplifying the processing procedure. In addition, the registration relationship between the at least one target region and the second image can be determined by simultaneously using the global registration technique and the local registration technique, which improves the accuracy of the determination.

In 506, the processor 102 (e.g., the positioning module 230) may determine the registration relationship between the at least one target region and the second image based on a corresponding relationship between the at least one reference point and the at least one target point.

In some embodiments, the processor 102 may obtain the position information of the at least one reference point and the at least one target point, and determine a transformation relationship between the position information of the at least one reference point and the at least one target point as the registration relationship. For example, the processor 102 may determine a transformation matrix between the at least one reference point and the at least one target point based on coordinates of the at least one reference point and the at least one target point. The transformation matrix may be represented as a transformation relationship between a coordinate system in which the at least one reference point is located and a coordinate system in which the at least one target point reference points is located. Further, the transformation matrix may also represent the registration relationship between the at least one target region and the second image.

It should be noted that the above descriptions of the process 500 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the process 500 may be accomplished with one or more additional operations not described, and/or without one or more of the operations discussed.

FIG. 9 is a block diagram illustrating an exemplary processor 102 according to some embodiments of the present disclosure. The processor 102 may include an obtaining module 910, a feature point determination module 920, and a posture determination module 930.

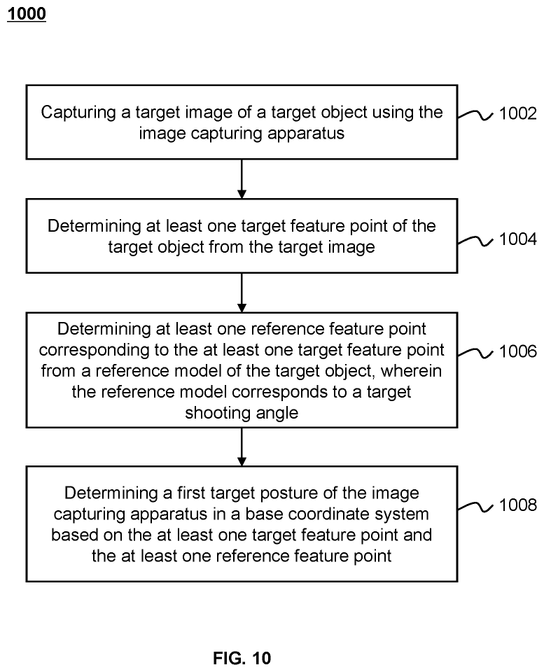

The obtaining module 910 may be configured to obtain a target image of a target object. The target image may include a 3D image (e.g., a depth image) and/or a 2D image of the target object. The target image may be captured using an image capturing apparatus. More descriptions regarding the obtaining the target image may be found in elsewhere in the present disclosure. See, e.g., operation 1002 in FIG. 10 and relevant descriptions thereof.

The feature point determination module 920 may be configured to determine at least one target feature point of the target object from the target image. A target feature point may represent a feature point of the target object in an image. In some embodiments, the feature point determination module 920 may also be configured to determine at least one reference feature point corresponding to the at least one target feature point from a reference model of the target object. The reference model may correspond to a target shooting angle. More descriptions regarding the determination of the at least one target feature point and the at least one reference feature point may be found in elsewhere in the present disclosure. See, e.g., operations 1004 and 1006 in FIG. 10 and relevant descriptions thereof.

The posture determination module 930 may be configured to determine a first target posture of the image capturing apparatus in a base coordinate system based on the at least one target feature point and the at least one reference feature point. In some embodiments, the first target posture may direct the image capturing apparatus to capture the target object from the target shooting angle and/or at a target shooting distance. More descriptions regarding the determination of the first target posture may be found in elsewhere in the present disclosure. See, e.g., operation 1008 in FIG. 10 and relevant descriptions thereof.

It should be noted that the descriptions of the robot control system and the modules thereof are provided for convenience of illustration, and are not intended to limit the scope of the present disclosure. It should be understood that those skilled in the art, having an understanding of the principles of the system, may arbitrarily combine the various modules or constitute subsystems connected to other modules without departing from the principles. For example, the obtaining module 910, the feature point determination module 920, and the posture determination module 930 disclosed in FIG. 9 may be different modules in the same system, or may be a single module that performs the functions of the modules mentioned above. As another example, modules of the robot control system may share a storage module, or each module may have an own storage module. As still another example, the obtaining module 910 and the obtaining module 210 may be the same module. Such modifications may not depart from the scope of the present disclosure.

In some embodiments, the processor 102 may further include a registration module configured to achieve registration between the target object and a planned image.

FIG. 10 is a flowchart illustrating an exemplary process 1000 for adjusting a posture of an image capturing apparatus according to some embodiments of the present disclosure. In some embodiments, the process 1000 may be implemented by the robot control system 100. For example, the process 1000 may be stored in a storage device (e.g., the storage 104) in the form of an instruction set (e.g., an application). In some embodiments, the processor 102 (e.g., the one or more modules as shown in FIG. 9) may execute the instruction set and direct one or more components of the robot control system 100 to perform the process 1000. In some embodiments, the adjustment of the posture of the image capturing apparatus described in operation 304 of FIG. 3 may be determined according to the process 1000.

In some embodiments, using a first image of a target object captured by the image capturing apparatus, the robot control system 100 may determine a surgical posture of the target object based on preoperative planning. By fixing the target object and adjusting the posture of the image capturing apparatus, a target region of the target object can be fully within a field of view of the image capturing apparatus.

At present, the image capturing apparatus is usually installed on a robot, and a doctor manually drags the image capturing apparatus to align with the target object. Since the doctor may not pay attention to physical characteristics of the image capturing apparatus during the drag process, it is difficult to adjust the image capturing apparatus quickly and accurately to an optimal posture, thereby reducing the efficiency of the shooting and the accuracy of the captured image data. In addition, the precision of the installation between the image capturing apparatus and the robot is reduced due to the drag of the image capturing apparatus by the doctor, further reducing the accuracy of the captured image data. Therefore, it is necessary to provide an effective system and method for adjusting the posture of the image capturing apparatus. In some embodiments, the posture of the image capturing apparatus may be adjusted by performing the following operations in the process 1000.

In 1002, the processor 102 (e.g., the obtaining module 910) may obtain a target image of a target object.

In some embodiments, the target object may include a biological object and/or a non-biological object. Merely by way of example, in a scenario of neurosurgery, the target object may be the head or face of a patient. The target image may include a 3D image (e.g., a depth image) and/or a 2D image of the target object. The target image may be captured using the image capturing apparatus. The target image is obtained may be in a similar manner to how the first image is obtained. More descriptions regarding the obtaining the target image may be found in elsewhere in the present disclosure. See, e.g., FIG. 3 and relevant descriptions thereof.

In 1004, the processor 102 (e.g., the feature point determination module 920) may determine at least one target feature point of the target object from the target image.

A target feature point may represent a feature point of the target object in an image. Merely by way of example, if the target object is the head or face of the patient, the at least one target feature point may be facial contour points of the target object as shown in FIG. 14. In some embodiments, the processor 102 may determine the at least one target feature point from the target image through a feature point extraction algorithm. For example, the processor 102 may determine the at least one target feature point from the target image through a feature point extraction model. More descriptions regarding the feature point extraction algorithm may be found in elsewhere in the present disclosure. See, e.g., operation 404 and relevant descriptions thereof.

In 1006, the processor 102 (e.g., the feature point determination module 920) may determine at least one reference feature point corresponding to the at least one target feature point from a reference model of the target object.

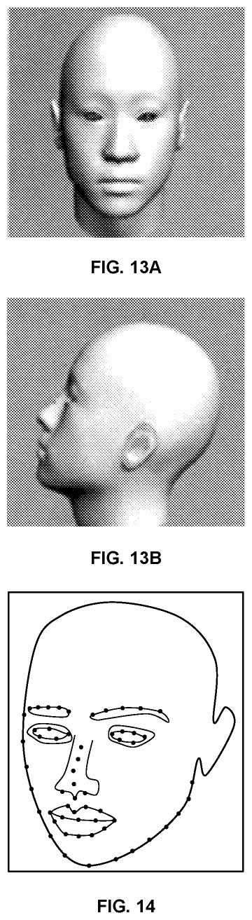

The reference model refers to a standard model that is constructed based on features of the target object. Merely by way of example, if the target object is the head or face of the patient, the reference model may be a standard human face model constructed based on head features of the human. Alternatively, the standard human face model may be downloaded from an open-source website. A front view and a side view of the standard human face model are shown in FIGS. 13A and 13B, respectively. In some embodiments, the reference model may be stored or displayed in the form of a 3D image, and the analysis and processing of the reference model may be performed based on the 3D image.

In some embodiments, the reference model may correspond to a target shooting angle. For example, the target shooting angle refers to an angle directly facing the target object. Merely by way of example, if the target object is the head or face of the patient, the target shooting angle may be an angle directly facing the face of the patient.

In some embodiments, each target feature point may correspond to a reference feature point. A target feature point and the corresponding reference feature point may correspond to a same physical point on the target object. The processor 102 may determine a corresponding reference feature point set from the reference model using the feature point extraction algorithm that is used to determine the at least one target feature point, and determine the reference feature point corresponding to each target feature point from the reference feature point set. In some embodiments, the processor 102 may determine at least one reference feature point from the reference model of the target object based on the at least one target feature point. For example, the processor 102 may determine the at least one reference feature point corresponding to the at least one target feature point based on a structural feature of the target object. As another example, the processor 102 may determine the at least one reference feature point corresponding to the at least one target feature point from the reference model of the target object using a machine learning model (e.g., a mapping model, an active appearance model (AAM), a MediaPipe model, etc.). Merely by way of example, the processor 102 may input the target image and the reference model into the mapping model, and the mapping model may output a mapping relationship between points in the target image and points in the reference model. The processor 102 may determine the at least one reference feature point corresponding to the at least one target feature point based on the mapping relationship.

In 1008, the processor 102 (e.g., the posture determination module 930) may determine a first target posture of the image capturing apparatus in a base coordinate system based on the at least one target feature point and the at least one reference feature point.

The base coordinate system may be any coordinate system. In some embodiments, the base coordinate system refers to a coordinate system established based on a base of a robot. For example, the base coordinate system may be established with a center of a base bottom of the robot as an origin, the base bottom as an XY plane, and a vertical direction as a Z axis.

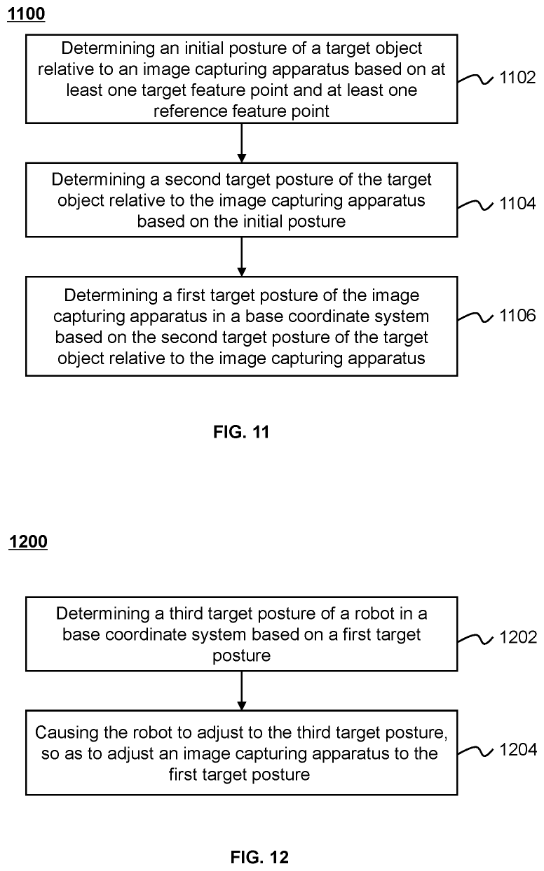

In some embodiments, the first target posture may reflect an adjusted posture of the image capturing apparatus in the base coordinate system. In some embodiments, the first target posture may direct the image capturing apparatus to capture the target object from the target shooting angle and/or at a target shooting distance. For example, the first target posture may be represented as a transformation relationship between a coordinate system (i.e., an updated first coordinate system) corresponding to the image capturing apparatus and the base coordinate system at the target shooting angle and target shooting distance. In some embodiments, the processor 102 may determine an initial posture of the target object relative to the image capturing apparatus based on the at least one target feature point and the at least one reference feature point. The processor 102 may determine a second target posture of the target object relative to the image capturing apparatus based on the initial posture. Further, the processor 102 may determine the first target posture of the image capturing apparatus in the base coordinate system based on the second target posture. More descriptions regarding the determination of the first target posture may be found in elsewhere in the present disclosure. See, e.g., FIG. 11 and relevant descriptions thereof.

In some embodiments, the image capturing apparatus may be installed on the robot. For example, the image capturing apparatus may be installed on an end terminal of a robotic arm of the robot. Therefore, the processor 102 may control the robot to move, so as to adjust the image capturing apparatus to the first target posture. In some embodiments, the processor 102 may determine a third target posture of the robot in the base coordinate system based on the first target posture. The processor 102 may control the robot to adjust to the third target posture, so as to adjust the image capturing apparatus to the first target posture. More descriptions regarding the determination of the third target posture may be found in elsewhere in the present disclosure. See, e.g., FIG. 12 and relevant descriptions thereof.

According to some embodiments of the present disclosure, the first target posture of the image capturing apparatus can be determined based on the at least one target feature point and the at least one reference feature point in the base coordinate system, so that the image capturing apparatus can shoot the target object at the target shooting angle and/or the target shooting distance. Therefore, the image capturing apparatus can be automatically positioned to the optimal position, which can improve the accuracy of the image data obtained by the image capturing apparatus, thereby improving the accuracy of subsequent robot positioning and preoperative planning.

It should be noted that the above descriptions of the process 1000 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the process 1000 may be accomplished with one or more additional operations not described, and/or without one or more of the operations discussed. For example, before capturing the target image of the target object, whether the field of view of the image capturing apparatus includes the target object may be determined. As another example, after adjusting the image capturing apparatus to the first target posture, image data (e.g., a first image) of the target object may be obtained for registering the target object with a planned image and/or positioning the robot.

FIG. 11 is a flowchart illustrating an exemplary process 1100 for determining a first target posture according to some embodiments of the present disclosure. In some embodiments, the process 1100 may be implemented by the robot control system 100. For example, the process 1100 may be stored in a storage device (e.g., the storage 104) in the form of an instruction set (e.g., an application). In some embodiments, the processor 102 (e.g., the one or more modules as shown in FIG. 9) may execute the instruction set and direct one or more components of the robot control system 100 to perform the process 1100. In some embodiments, the first target posture described in operation 1008 of FIG. 10 may be determined according to the process 1100.

In 1102, the processor 102 (e.g., the posture determination module 930) may determine an initial posture of a target object relative to an image capturing apparatus based on at least one target feature point and at least one reference feature point.

In some embodiments, the initial posture may be represented as a transformation relationship between a coordinate system corresponding to the target object and a first coordinate system corresponding to the image capturing apparatus when capturing a target image. In some embodiments, the initial posture may reflect the initial posture of the target object in the first coordinate system when the image capturing apparatus is at a target shooting angle, and/or an adjustment angle needed for adjusting the target object to a posture corresponding to the reference model in the first coordinate system. In some embodiments, the initial posture may be represented as coordinates of the image capturing apparatus in the first coordinate system corresponding to the image capturing apparatus when capturing the target image.

In some embodiments, the processor 102 may transform the issue of determining the initial posture of the target object in the first coordinate system into solving a Perspective N Points (PNP) problem. The PNP problem refers to an object positioning problem that assumes that the image capturing apparatus is a pinhole model and has been calibrated, an image of N space points whose coordinates in the object coordinate system are known is taken, and coordinates of the N space points in the image are known, and the object positioning problem is used to determine the coordinates of the N space points in the first coordinate system corresponding to the image capturing apparatus.

Merely by way of example, the at least one target feature point may be facial contour points of the target object. After the facial contour points of the target object are determined, coordinates of the facial contour points in a coordinate system corresponding to the target object may be obtained. The coordinate system corresponding to the target object may be established based on the target object. Taking a human face as an example, the tip of the nose may be an origin of a face coordinate system, a plane parallel to the face may be an XY plane, and a direction perpendicular to the face may be a Z-axis. For example, if the target image is a 2D image, pixel coordinates corresponding to n determined facial contour points may be denoted as Aᵢ(xᵢ, yᵢ) (i=1, 2, 3, . . . , n), and spatial coordinates of n reference feature points in a reference model corresponding to the facial contour points in the coordinate system corresponding to the target object may be denoted as Bⱼ(Xⱼ, Yⱼ, Zⱼ) (j=1, 2, 3, . . . , n). The initial posture

$${face}_{camera}^{} T$$

of the target object relative to the image capturing apparatus before the posture adjustment (also referred to as a posture transformation matrix

$${face}_{camera}^{} T$$

from the coordinate system corresponding to the target object to the first coordinate system) may be determined according to Equation (1):

$$[ \begin{matrix}{x}_{i} \\ {y}_{i} \\ 1\end{matrix} ] = M {}_{face}^{camera} T [ \begin{matrix}{X}_{j} \\ {Y}_{j} \\ {Z}_{j}\end{matrix} ] ( i = j = 1 , 2 , 3 … n ) , \qquad ( 1 )$$

where M refers to a parameter matrix of the image capturing apparatus, which may be determined based on intrinsic parameter(s) of the image capturing apparatus.

As another example, if the target image is a 3D image, pixel coordinates corresponding to n determined facial contour points may be denoted as Aᵢ(xᵢ, yᵢ, zᵢ) (i=1, 2, 3, . . . , n), and spatial coordinates of n reference feature points in a reference model corresponding to the facial contour points in the coordinate system corresponding to the target object may be denoted as Bⱼ(Xⱼ, Yⱼ, Zⱼ) (j=1, 2, 3, . . . , n). The initial posture

$${face}_{camera}^{} T$$

the target object relative to the image capturing apparatus before the posture adjustment (also referred to as a posture transformation matrix

$${face}_{camera}^{} T$$

from the coordinate system corresponding to the target object to the first coordinate system) may be determined according to Equation (2):

$$[ \begin{matrix}{x}_{i} \\ {y}_{i} \\ {z}_{i}\end{matrix} ] = M {}_{face}^{camera} T [ \begin{matrix}{X}_{j} \\ {Y}_{j} \\ {Z}_{j}\end{matrix} ] ( i = j = 1 , 2 , 3 … n ) . \qquad ( 2 )$$

Through the above operation, the angle needed for adjusting the target object to the posture corresponding to the reference model in the first coordinate system may be determined based on the at least one target feature point, the at least one reference feature point, and the parameter matrix of the image capturing apparatus.

In 1104, the processor 102 (e.g., the posture determination module 930) may determine a second target posture of the target object relative to the image capturing apparatus based on the initial posture.

The second target posture may be represented as a transformation relationship between the coordinate system corresponding to the target object and an updated first coordinate system corresponding to the adjusted image capturing apparatus. In some embodiments, the second target posture may reflect a posture of the target object in the updated first coordinate system after adjusting the posture of the image capturing apparatus (e.g., adjusting to the target shooting angle and the target shooting distance), and/or a distance needed for adjusting a shooting distance to the target shooting distance in the updated first coordinate system.

The target shooting distance refers to a distance in a height direction between the target object and the image capturing apparatus when the quality of the image data captured by the image capturing apparatus meets a preset standard. In some embodiments, the processor 102 may obtain the target shooting distance of the image capturing apparatus. For example, the target shooting distance may be predetermined and stored in a storage device (e.g., the storage 104), and the processor 102 may retrieve the target shooting distance from the storage device. Merely by way of example, when the whole target object (or other reference objects, such as a facial model) is displayed within the field of view of the image capturing apparatus, the image capturing apparatus may be moved along a direction of an optical axis of the image capturing apparatus to capture image data of the target object at a plurality of shooting distances. The processor 102 may determine an optimal shooting distance between the target object and the image capturing apparatus based on the accuracy and quality (e.g., definition) of the image data at each of the plurality of shooting distances, and determine the optimal shooting distance as the target shooting distance. In some embodiments, the target shooting distance of the image capturing apparatus may be determined through marker points. For example, a plurality of marker points may be disposed on the target object, and coordinates of the plurality of marker points may be determined as standard coordinates through a high-precision image capturing device. The processor 102 may determine coordinates of the plurality of marker points at the different shooting distances, respectively, by capturing the plurality of marker points at different shooting distances using the image capturing apparatus. The processor 102 may compare the coordinates determined at the different shooting distances with the standard coordinates, and determine a shooting distance corresponding to coordinates with a minimum deviation from the standard coordinates as the target shooting distance.

In some embodiments, the processor 102 may determine a distance transformation matrix based on the target shooting distance. Merely by way of example, if the target shooting distance is H, the distance transformation matrix P may be represented as shown in Equation (3):

$$P = [ \begin{matrix}1 & 0 & 0 & 0 \\ 0 & 1 & 0 & 0 \\ 0 & 0 & 1 & H \\ 0 & 0 & 0 & 1\end{matrix} ] . \qquad ( 3 )$$

In some embodiments, the processor 102 may determine the second target posture (i.e., a target posture of the target object relative to the image capturing apparatus after the posture adjustment) of the target object in the updated first coordinate system at the target shooting distance based on the distance transformation matrix and the initial posture. For example, the second target posture may be determined according to Equation (4):

$${}_{face}^{camera} {T}^{′} = {}_{face}^{camera} T \times [ \begin{matrix}1 & 0 & 0 & 0 \\ 0 & 1 & 0 & 0 \\ 0 & 0 & 1 & H \\ 0 & 0 & 0 & 1\end{matrix} ] , \qquad ( 4 )$$

where

$${face}_{camera}^{} T$$

refers to the initial posture of the target object in the first coordinate system before the update, and

$${face}_{camera}^{} {T}^{′}$$

refers to the second target posture of the target object in the updated first coordinate system at the target shooting distance.

By determining the second target posture of the target object in the updated first coordinate system at the target shooting distance, the shooting distance between the target object and the image capturing apparatus can be adjusted to the target shooting distance, thereby improving the accuracy of the image data captured by the image capturing apparatus.

In 1106, the processor 102 (e.g., the posture determination module 930) may determine a first target posture of the image capturing apparatus in a base coordinate system based on the second target posture of the target object relative to the image capturing apparatus.

In some embodiments, the image capturing apparatus may be installed on a robot. Therefore, a fourth transformation relationship between the first coordinate system and the base coordinate system may be determined through a connection structure between the image capturing apparatus and the robot. Further, the processor 102 may determine the first target posture based on the fourth transformation relationship and the second target posture.

Merely by way of example, the processor 102 may obtain a first transformation relationship between the first coordinate system and a second coordinate system corresponding to the robot. The first transformation relationship refers to a mapping relationship between a position of the robot and a position of the image capturing apparatus. More descriptions regarding the obtaining the first transformation relationship may be found in elsewhere in the present disclosure. See, e.g., FIG. 3 and relevant descriptions thereof. The processor 102 may also obtain a fifth transformation relationship between the second coordinate system and the base coordinate system. In some embodiments, the processor 102 may determine the fifth transformation relationship through a preset calibration technique (e.g., a hand-eye calibration technique). For example, the processor 102 may determine the fifth transformation relationship in a similar manner to how the first transformation relationship is determined. In some embodiments, the fifth transformation relationship may be a parameter of the robot, and the processor 102 may retrieve the fifth transformation relationship from a controller of the robot. Further, the processor 102 may determine the fourth transformation relationship based on the first transformation relationship and the fifth transformation relationship. Merely by way of example, the fourth transformation relationship may be determined according to Equation (5):

$$T = {}_{camera}^{tool} T \times {}_{tool}^{base} T , \qquad ( 5 )$$

wherein T refers to the fourth transformation relationship between the first coordinate system corresponding to the image capturing apparatus and the base coordinate system,

$${}_{camera}^{tool} T$$

refers transformation relationship between the first coordinate system corresponding to the image capturing apparatus and the second coordinate system corresponding to the robot, and

$${}_{tool}^{base} T$$

refers to the fifth transformation relationship between the second coordinate system corresponding to the robot and the base coordinate system.

In some embodiments, the processor 102 may determine the first target posture based on the fourth transformation relationship and the second target posture. Merely by way of example, the first target posture may be determined according to Equation (6):

$${camera}_{base}^{} {T}^{′} = {}_{face \_ link \_ view}^{base} T = {}_{face}^{camera} {T}^{′} \times {}_{camera}^{tool} T \times {}_{tool}^{base} T , \qquad ( 6 )$$

where

$${camera}_{base}^{} {T}^{′} and {}_{face \_ link \_ view}^{base} T$$

refer to the first target posture.

According to some embodiments of the present disclosure, by determining the fourth transformation relationship between the first coordinate system corresponding to the image capturing apparatus and the base coordinate system, the first target posture of the target object in the base coordinate system can be determined based on the fourth transformation relationship and the second target posture of the target object in the updated first coordinate system, which can adjust the image capturing apparatus to the target shooting angle and the target shooting distance, thereby improving the accuracy of the captured image data.

It should be noted that the above descriptions of the process 1100 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the process 1100 may be accomplished with one or more additional operations not described, and/or without one or more of the operations discussed.

FIG. 12 is a flowchart illustrating an exemplary process 1200 for adjusting a posture of an image capturing apparatus according to some embodiments of the present disclosure. In some embodiments, the process 1200 may be implemented by the robot control system 100. For example, the process 1200 may be stored in a storage device (e.g., the storage 104) in the form of an instruction set (e.g., an application). In some embodiments, the processor 102 (e.g., the one or more modules as shown in FIG. 9) may execute the instruction set and direct one or more components of the robot control system 100 to perform the process 1200.

In some embodiments, an image capturing apparatus may be installed on a robot. For example, the image capturing apparatus may be installed at an end terminal of a robotic arm of the robot. The process 1200 may be performed after operation 1008 described in FIG. 10, so that the robot control system 100 can control the movement of the robot to adjust the image capturing apparatus to the first target posture.

In 1202, the processor 102 (e.g., the posture determination module 930) may determine a third target posture of a robot in a base coordinate system based on a first target posture.

In some embodiments, the third target posture may reflect a posture of the robot in the base coordinate system when the image capturing apparatus is in the first target posture, and/or a distance and an angle that the robot needs to move in the base coordinate system to adjust the image capturing apparatus to the first target posture. In some embodiments, the third target posture may be represented as a transformation relationship between a second coordinate system corresponding to the robot and the base coordinate system when the image capturing apparatus is in the first target posture.

In some embodiments, the processor 102 may determine the third target posture of the robot in the base coordinate system based on a first transformation relationship and the first target posture. Merely by way of example, the third target posture may be determined according to Equation (7):

$${tool}_{base}^{} {T}^{′} {=}_{camera}^{base} {T}^{′} \times {( {}_{camera}^{tool} T )}^{- 1} , \qquad ( 7 )$$

where

$${tool}_{base}^{} {T}^{′}$$

refers to the third target posture.

In 1204, the processor 102 (e.g., the posture determination module 930) may cause the robot to adjust to the third target posture, so as to adjust the image capturing apparatus to the first target posture.

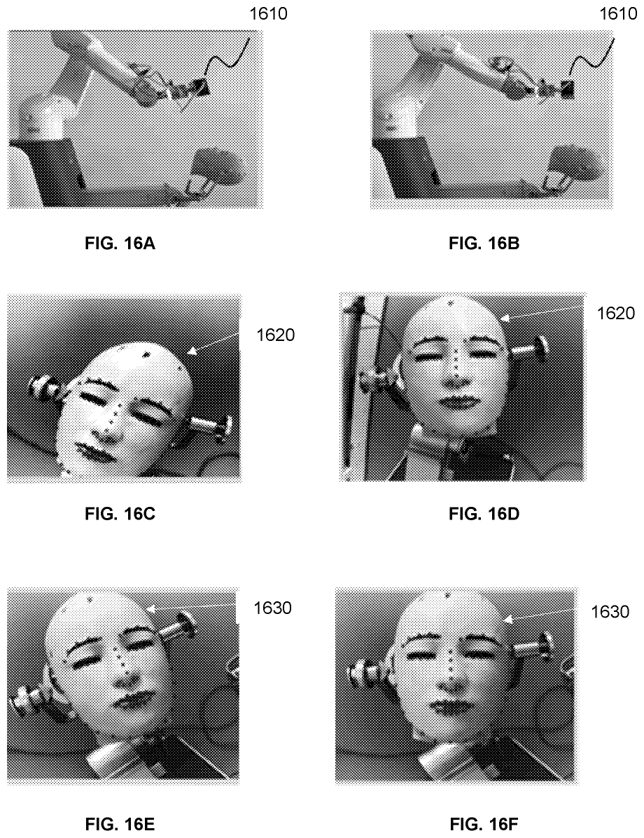

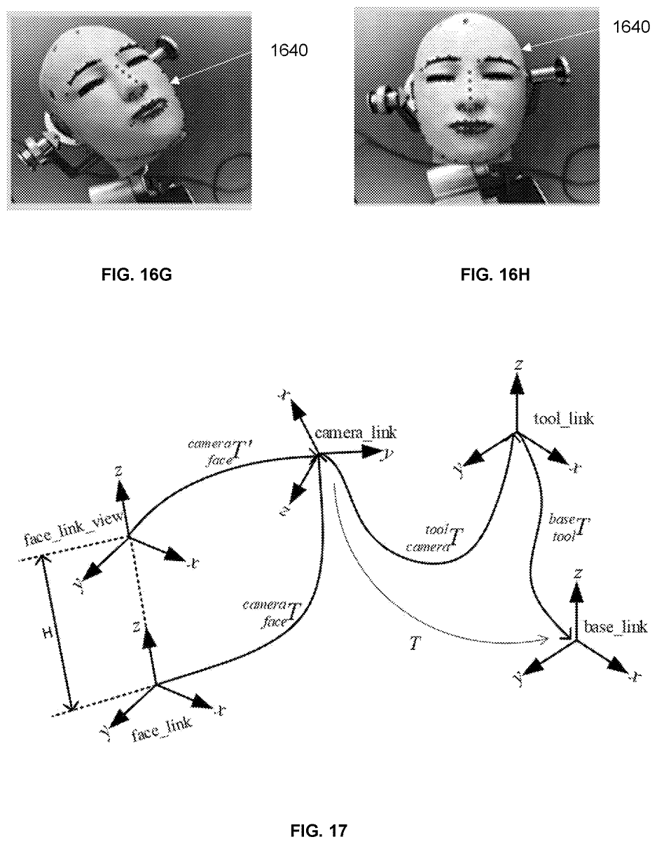

Merely by way of example, referring to FIGS. 16A to 16H, FIG. 16A is a schematic diagram illustrating an image capturing apparatus before posture adjustment according to some embodiments of the present disclosure. FIG. 16B is a schematic diagram illustrating an image capturing apparatus after posture adjustment according to some embodiments of the present disclosure. FIGS. 16C, 16E, and 16G are schematic diagrams illustrating image data captured by an image capturing apparatus before posture adjustment according to some embodiments of the present disclosure. FIGS. 16D, 16F, and 16H are schematic diagrams illustrating image data captured by an image capturing apparatus after posture adjustment according to some embodiments of the present disclosure. Combining FIGS. 16A and 16B, by causing a robot to adjust to a third target posture, an image capturing apparatus 1610 is adjusted from a posture as shown in FIG. 16A to a first target posture as shown in FIG. 16B. Comparing FIGS. 16C and 16D, FIGS. 16E and 16F, and FIGS. 16G and 16H, it may be determined that imaging positions of target objects 1620, 1630, and 1640 in image data are adjusted to a center of the image data, respectively, after the image capturing apparatus is adjusted to the first target posture. In other words, after the image capturing apparatus is adjusted to the first target posture, the image capturing apparatus can capture the image data of the target objects at a target shooting angle and a target shooting height.

According to some embodiments of the present disclosure, the posture (i.e., the third target posture) of the robot in the base coordinate system can be determined accurately, thereby accurately adjusting the posture of the image capturing apparatus based on the posture of the robot in the base coordinate system. Therefore, the image capturing apparatus can capture the image data of the target objects at the target shooting angle and the target shooting distance, thereby improving the accuracy of the captured image data.

It should be noted that the above descriptions of the process 1200 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the process 1200 may be accomplished with one or more additional operations not described, and/or without one or more of the operations discussed.

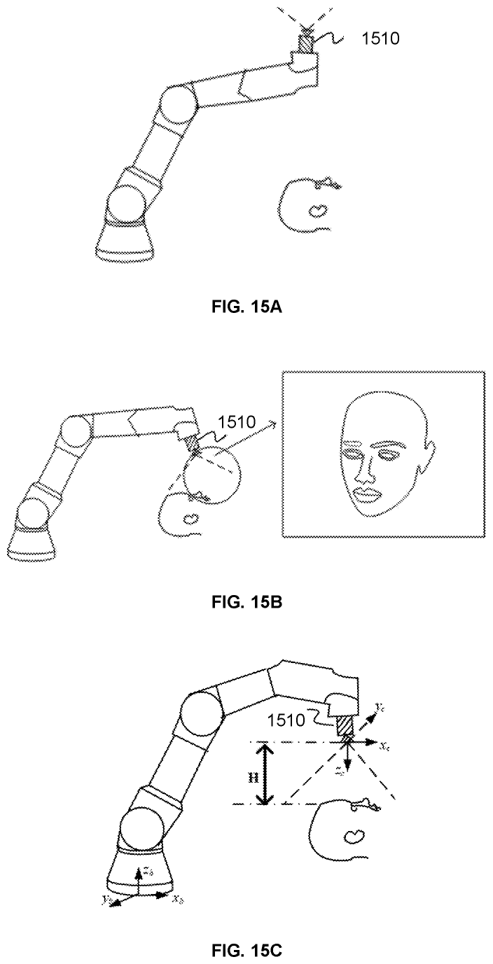

FIGS. 15A to 15C are schematic diagrams illustrating an exemplary process for adjusting a posture of an image capturing apparatus 1510 according to some embodiments of the present disclosure.

After the image capturing apparatus 1510 is installed, a robot may be located at a random initial position. As shown in FIG. 15A, a field of view of the image capturing apparatus 1510 does not include the face of a patient (i.e., a target object). Therefore, the posture of the robot needs to be adjusted locally first, so that the field of view of the image capturing apparatus 1510 displays the whole or partial face of the patient. Merely by way of example, before capturing a target image of the target object, the robot control system 100 may determine whether the field of view of the image capturing apparatus 1510 includes at least one target feature point. In response to determining that the field of view of the image capturing apparatus 1510 includes the at least one target feature point, the robot control system 100 may capture the target image of the target object using the image capturing apparatus 1510. In response to determining that the field of view of the image capturing apparatus 1510 does not include the at least one target feature point, the robot control system 100 may adjust the image capturing apparatus 1510, so that the field of view of the image capturing apparatus 1510 includes the at least one target feature point.

When the field of view of the image capturing apparatus 1510 includes the at least one target feature point (as shown in FIG. 15B), the robot control system 100 (e.g., the processor 102) may execute a process for adjusting a posture of the image capturing apparatus as illustrated in FIGS. 10 to 12, so that the image capturing apparatus 1510 can capture image data of the target object from a target shooting angle and a target shooting distance (as shown in FIG. 15C).

By adjusting the image capturing apparatus to include at least a portion of the target object in the field of view of the image capturing apparatus before capturing the target image, the efficiency of adjusting the posture of the image capturing apparatus can be improved.

FIG. 17 is a schematic diagram illustrating an exemplary posture adjustment process of an image capturing apparatus according to some embodiments of the present disclosure.

As shown in FIG. 17, a first coordinate system corresponding to an image capturing apparatus when capturing a target image is camera_link, a coordinate system corresponding to a target object is face_link, an updated first coordinate system of the image capturing apparatus at a target shooting distance is face_link_view, a second coordinate system corresponding to a robot is tool_link, and a base coordinate system is base_link.

An initial posture

$${}_{face}^{camera} T$$

of the target object in the first coordinate system camera_link may be determined, based on at least one target feature point and at least one reference feature point, using Equation (1) or Equation (2). A second target posture

$${}_{face}^{camera} {T}^{′}$$

of the target object in the updated first coordinate system face_link_view at the target shooting distance may be determined using Equation (4). By obtaining a first transformation relationship

$${}_{camera}^{tool} T$$

between the first coordinate system camera_link and the second coordinate system tool_link and obtaining a fifth transformation relationship

$${}_{tool}^{base} T$$

between the second coordinate system tool_link and the base coordinate system base_link, a fourth transformation relationship T between the first coordinate system camera_link and the base coordinate system base_link may be determined using Equation (5). The first target posture

$${camera}_{base}^{} {T}^{′}$$

may be determined, based on the fourth transformation relationship T and the second target posture

$${}_{face}^{camera} {T}^{′} ,$$

using Equation (6).

In some embodiments, a third target posture of the robot in the base coordinate system may be determined, based on an inverse operation

$${( {}_{camera}^{tool} T )}^{- 1}$$

of the first transformation relationship and the first target posture

$${camera}_{base}^{} {T}^{′} ,$$

using Equation (7). Merely by way of example,

$${tool}_{base}^{} {T}^{′}$$

may be determined using Equation (7).

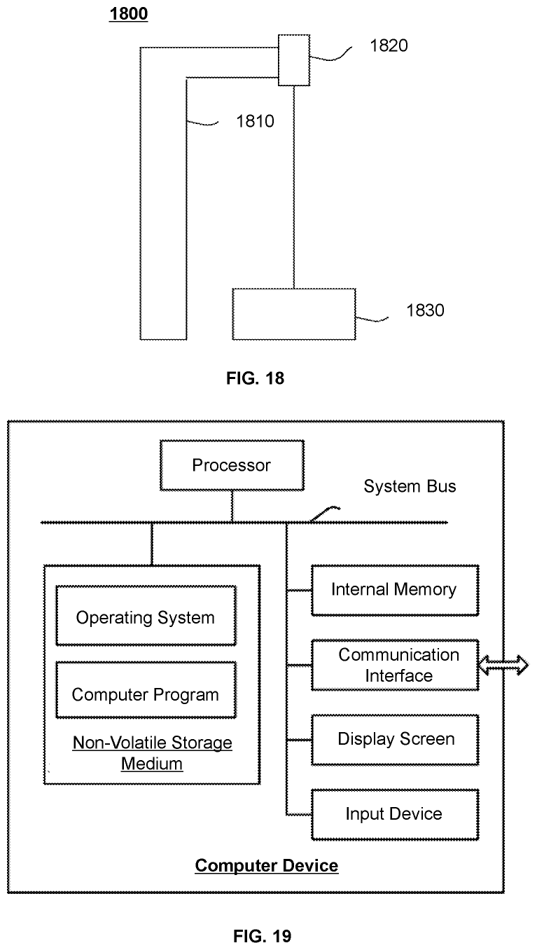

FIG. 18 is a schematic diagram illustrating an exemplary robot control system 1800 according to some embodiments of the present disclosure.

As shown in FIG. 18, the robot control system 1800 may include a robot 1810, an image capturing apparatus 1820, and a processor 1830. The image capturing apparatus 1820 may be installed on the robot 1810 (e.g., at an end terminal of a robotic arm of the robot). The processor 1830 may be connected to the robot 1810 and the image capturing apparatus 1820, respectively. When the processor 1830 operates, the processor 1830 may execute the robot positioning process and the posture adjustment process of the image capturing apparatus as described in some embodiments of the present disclosure.

In one embodiment, a computer device is provided. The computer device may be a server, and an internal structure diagram of the computer device may be as shown in FIG. 19. The computer device includes a processor, a storage, a communication interface, a display screen, and an input device connected via a system bus. The processor of the computer device is configured to provide computation and control capabilities. For example, the processor of the computer device may execute the robot positioning method and posture adjustment method of image capturing apparatus as described in some embodiments of the present disclosure. The storage of the computer device includes a non-volatile storage medium and an internal memory. The non-volatile storage medium stores an operating system and a computer program. The internal memory provides an environment for running the operating system and the computer program stored in the non-volatile storage medium. The communication interface of the computer device is used for wired or wireless communication with an external terminal. The wireless communication may be achieved through techniques, such as Wi-Fi, a cellular network, a near field communication (NFC), etc. The computer program is executed by the processor to implement the robot positioning method. The display screen of the computer device may include an LCD screen or an e-ink display screen. The input device of the computer device may include a touch layer overlaid on the display screen, a physical button, a trackball, or a touchpad on the housing of the computer device, or an external device such as a keyboard, a touchpad, a mouse, etc.

It should be understood by those skilled in the art that the structure shown in FIG. 19 is merely a block diagram of a partial structure related to the embodiments of the present disclosure, and does not constitute a limitation on the computer device to which the embodiments of the present disclosure are applied. A specific computer device may include more or fewer parts than the structure shown in the FIG. 19, or combine certain components, or include a different arrangement of components.

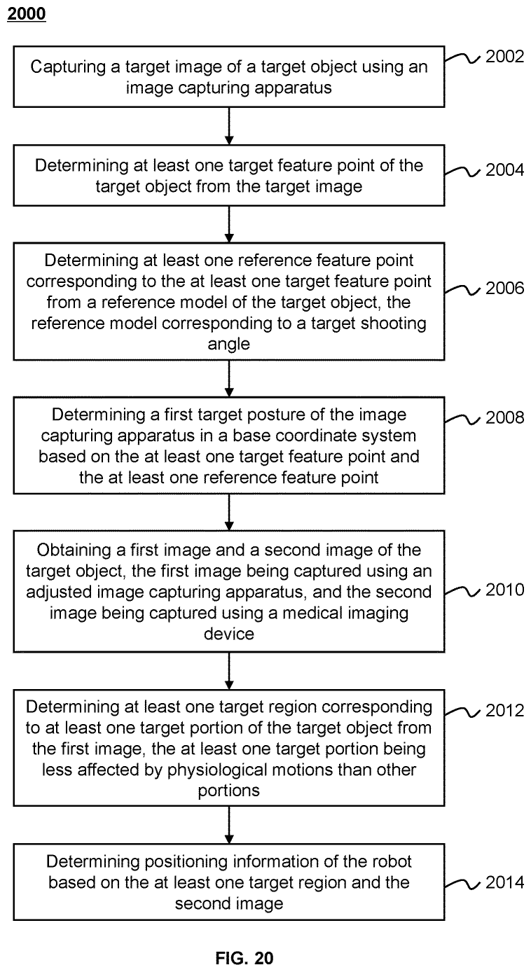

FIG. 20 is a flowchart illustrating an exemplary robot control process 2000 according to some embodiments of the present disclosure. In some embodiments, the process 2000 may be implemented by the robot control system 100. For example, the process 2000 may be stored in a storage device (e.g., the storage 104) in the form of an instruction set (e.g., an application). In some embodiments, the processor 102 (e.g., the one or more modules as shown in FIG. 2 and/or the one or more modules shown in FIG. 9) may execute the instruction set and direct one or more components of the robot control system 100 to perform the process 2000.

In 2002, the processor 102 may capture a target image of a target object using an image capturing apparatus.

In 2004, the processor 102 may determine at least one target feature point of the target object from the target image.

In 2006, the processor 102 may determine at least one reference feature point corresponding to the at least one target feature point from a reference model of the target object. The reference model may correspond to a target shooting angle.

In 2008, the processor 102 may determine a first target posture of the image capturing apparatus in a base coordinate system based on the at least one target feature point and the at least one reference feature point, such that the image capturing apparatus can shoot the target object from the target shooting angle.

In 2010, the processor 102 may obtain a first image and a second image of the target object. The first image may be captured using an adjusted image capturing apparatus, and the second image may be captured using a medical imaging device.

In 2012, the processor 102 may determine at least one target region corresponding to at least one target portion of the target object from the first image. The at least one target portion may be less affected by physiological motions than other portions.

In 2014, the processor 102 may determine positioning information of the robot based on the at least one target region and the second image.

According to some embodiments of the present disclosure, a posture adjustment may be performed on the image capturing apparatus, and then a positioning operation is performed on the robot. The image capturing apparatus after the posture adjustment can capture the first image at the target shooting angle and the target shooting distance, which can improve the accuracy of the first image, thereby improving the accuracy of the robot positioning, and the accuracy of preoperative planning or surgical operations.

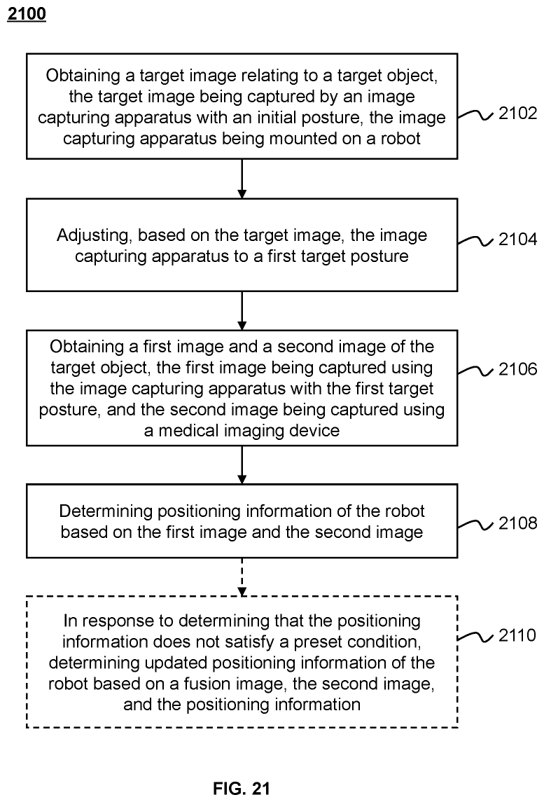

FIG. 21 is a flowchart illustrating an exemplary process 2100 for robot positioning according to some embodiments of the present disclosure. In some embodiments, the process 2100 may be implemented by the robot control system 100. For example, the process 2100 may be stored in a storage device (e.g., the storage 104) in the form of an instruction set (e.g., an application). In some embodiments, the processor 102 (e.g., the one or more modules as shown in FIG. 2 and/or FIG. 9) may execute the instruction set and direct one or more components of the robot control system 100 to perform the process 2100.

In 2102, the processor 102 (e.g., the obtaining module 910) may obtain a target image relating to a target object.

In some embodiments, the target object may include a biological object and/or a non-biological object. Merely by way of example, in a scenario of neurosurgery, the target object may be the head or face of a patient.

The target image relating to the target object may be an image of the target object and/or an environmental image of the environment where the target object is located. For example, the target image may include a 3D image (e.g., a depth image) of the target object or the environment where the target object is located. In some embodiments, the target image may also be referred to as first point cloud data. In some embodiments, the target image may be captured by an image capturing apparatus (e.g., the image capturing apparatus 130, the image capturing apparatus 1820) with an initial posture, and used to adjust the capturing apparatus to a desired posture (e.g., the first target posture). For example, the depth image are captured by the image capturing apparatus with the initial posture. The initial posture may be represented as a transformation relationship between a coordinate system corresponding to the target object and a first coordinate system corresponding to the image capturing apparatus when capturing the target image. In some embodiments, the initial posture may be represented as coordinates of the image capturing apparatus in the first coordinate system corresponding to the image capturing apparatus when capturing the target image. More descriptions regarding the initial posture may be found elsewhere in the present disclosure. See, e.g., FIGS. 11 and 17 and relevant descriptions thereof.

In some embodiments, the image capturing apparatus may be mounted on a robot (e.g., the robot 140, the robot 1810). For example, the image capturing apparatus may be mounted (or installed) on an end terminal of a robotic arm of the robot. More descriptions regarding the mounting of the image capturing apparatus may be found elsewhere in the present disclosure. See, e.g., FIGS. 12 and 18 and relevant descriptions thereof. In some embodiments, operation 2102 may be performed in a similar manner as operation 2002 as described in connection with FIG. 20.

In some embodiments, the processor 102 may further obtain a second 2D reference image (e.g., a 2D RGB image) of the target object captured by the image capturing apparatus with the initial posture. For example, the second 2D reference image and the target image may be captured by the image capturing apparatus simultaneously.

In 2104, the processor 102 (e.g., the posture determination module 930) may adjust, based on the target image, the image capturing apparatus to a first target posture.

The first target posture may be in a base coordinate system corresponding to the robot. The base coordinate system may be any coordinate system. For example, the base coordinate system refers to a coordinate system established based on a base of a robot. More descriptions regarding the base coordinate system may be found elsewhere in the present disclosure. See, e.g., FIG. 11 and relevant descriptions thereof.

In some embodiments, the first target posture may reflect an adjusted posture of the image capturing apparatus in the base coordinate system. In some embodiments, the first target posture may direct the image capturing apparatus to capture the target object from a target shooting angle and/or at a target shooting distance. The target shooting angle refers to an angle directly facing the target object. For example, if the target object is the head or face of the patient, the target shooting angle may be an angle directly facing the face of the patient. The target shooting distance refers to a distance in a height direction between the target object and the image capturing apparatus when the quality of the image data captured by the image capturing apparatus meets a preset standard. Merely by way of example, the first target posture may be represented as a transformation relationship between a coordinate system (i.e., an updated first coordinate system) corresponding to the image capturing apparatus and the base coordinate system at the target shooting angle and/or the target shooting distance. More descriptions regarding the first target posture, the target shooting angle, and the target shooting distance may be found elsewhere in the present disclosure. See, e.g., FIG. 11 and relevant descriptions thereof.

In some embodiments, when the target image is an image (e.g., the depth image) of the target object, the processor 102 may determine the first target posture (e.g., the target shooting angle and/or the target shooting distance) of the image capturing apparatus based on the target image. For example, the processor 102 may determine the first target posture of the image capturing apparatus based on the target image and a reference model of the target object, and the reference model refers to a standard model that is constructed based on features of the target object. As another example, the processor 102 may determine a second target posture of the target object relative to the image capturing apparatus based on the initial posture, determine a fourth transformation relationship between the first coordinate system corresponding to the image capturing apparatus and the base coordinate system, and determine the first target posture based on the second target posture and the fourth transformation relationship. More descriptions regarding the determination of the target shooting angle and/or the target shooting distance based on the target image may be found elsewhere in the present disclosure. See, e.g., FIGS. 11 and 17 and relevant descriptions thereof.

In some embodiments, the processor 102 may determine the posture of the target object (e.g., the position and the orientation of the face) in the first coordinate system based on the target image. Based on the determined posture of the target object, the processor 102 may determine a posture of the image capturing apparatus in the first coordinate system at which the image capturing apparatus can capture the target subject from the target shooting angle and/or the target shooting distance. The processor 102 may further transform the posture of the image capturing apparatus in the first coordinate system into the first target posture in the base coordinate system based on the fourth transformation relationship.

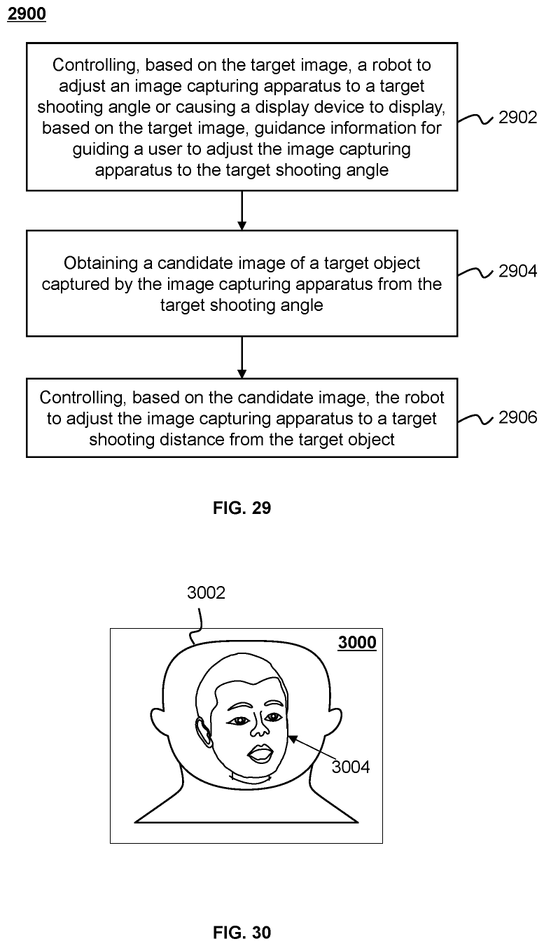

In some embodiments, the processor 102 may determine the target shooting angle based on the target image, and then control the robot to adjust the image capturing apparatus to the target shooting angle or causing a display device (e.g., the display device of the input/output device 108) to display, based on the target image, guidance information for guiding a user to adjust the image capturing apparatus to the target shooting angle. Further, the processor 102 may obtain a candidate image of the target object captured by the image capturing apparatus from the target shooting angle, and control the robot to adjust the image capturing apparatus to the target shooting distance from the target object based on the candidate image. More descriptions regarding the adjustment of the image capturing apparatus may be found elsewhere in the present disclosure. See, e.g., FIG. 29 and relevant descriptions thereof.

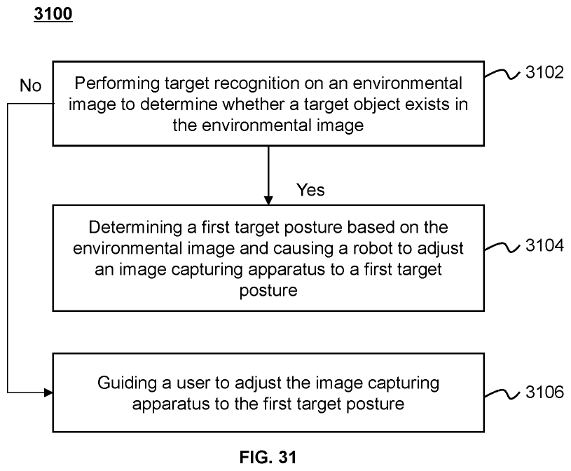

In some embodiments, when the target image is the environmental image of the environment where the target object is located, the processor 102 may perform target recognition on the environmental image to determine whether the target object exists in the environmental image. If the target object exists in the environmental image, the processor 102 may determine the first target posture based on the environmental image and cause the robot to adjust the image capturing apparatus to the first target posture. If the target object does not exist in the environmental image, the processor 102 may guide the user to adjust the image capturing apparatus to the first target posture. More descriptions regarding the adjustment of the image capturing apparatus may be found elsewhere in the present disclosure. See, e.g., FIG. 31 and relevant descriptions thereof.

In some embodiments, the image capturing apparatus may be manually adjusted by the user. For example, the user may adjust the image capturing apparatus to the first target posture based on the guidance information. As another example, the image capturing apparatus may be mounted on the end terminal of the robotic arm of the robot, and the user may adjust the end terminal of the robotic arm to move the image capturing apparatus to the first target posture. In this case, the image capturing apparatus may be adjusted in a manual manner.

In some embodiments, the image capturing apparatus may be adjusted by the robot. For example, the processor 102 may control the robot to move, so as to adjust the image capturing apparatus to the first target posture. In this case, the image capturing apparatus may be adjusted in an automatic manner.

In some embodiments, the manual manner and the automatic manner may be combined to adjust the image capturing apparatus. That is, a semi-automatic manner may be used to adjust the image capturing apparatus. For example, the user may adjust the image capturing apparatus to the target shooting angle, and the processor 102 may control the robot to adjust the image capturing apparatus to the target shooting distance. More descriptions regarding the semi-automatic manner may be found elsewhere in the present disclosure. See, e.g., FIG. 29 and relevant descriptions thereof.

In some embodiments, during the adjustment of the image capturing apparatus to the first target posture, the processor 102 may control the image capturing apparatus to obtain one or more second candidate images. The one or more second candidate images may be configured to determine a real-time posture of the image capturing apparatus/the robot. This can improve the accuracy of the adjustment of the image capturing apparatus to the first target posture.

In 2106, the processor 102 (e.g., the obtaining module 210) may obtain a first image and a second image of the target object. The first image may be captured using the image capturing apparatus with the first target posture, and the second image may be captured using a medical imaging device.

The first image refers to an image obtained using the image capturing apparatus (e.g., the image capturing apparatus 130) with the first target posture. For example, the first image may be a 3D image, such as a depth image, of the target object captured using the image capturing apparatus with the first target posture. The second image refers to a medical image captured using a medical imaging device (e.g., the medical imaging device 120). More descriptions regarding the first image and the second image may be found elsewhere in the present disclosure. See, e.g., FIG. 3 and relevant descriptions thereof.

In some embodiments, the processor 102 may further obtain a 2D reference image (also referred to as a first 2D reference image) of the target object captured using the image capturing apparatus. More descriptions regarding the first 2D reference image may be found elsewhere in the present disclosure. See, e.g., FIG. 4 and relevant descriptions thereof.

In 2108, the processor 102 (e.g., the positioning module 230) may determine positioning information of the robot based on the first image and the second image.

In some embodiments, operation 2108 may be performed in a similar manner as operation 306.

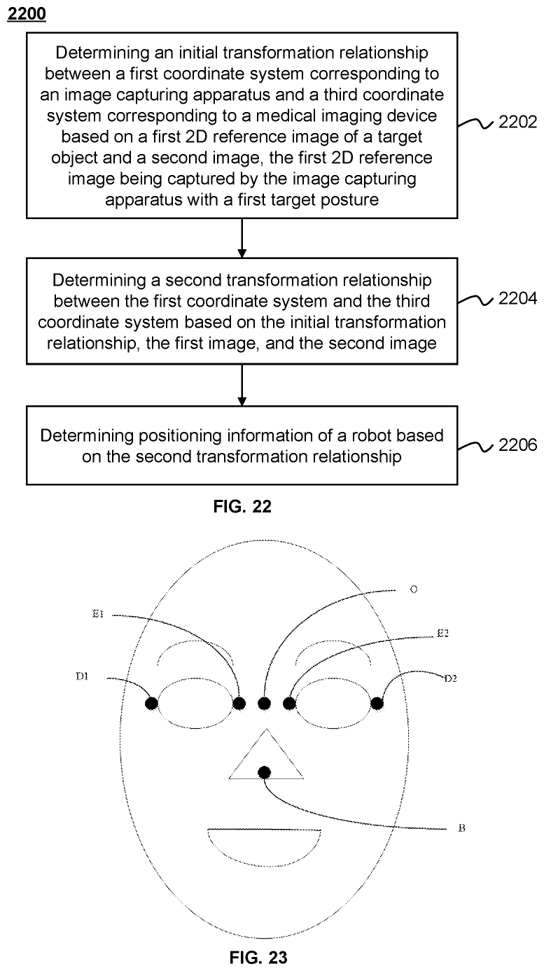

In some embodiments, the processor 102 may determine an initial transformation relationship between the first coordinate system corresponding to the image capturing apparatus and the third coordinate system corresponding to the medical imaging device based on the first 2D reference image of the target object and the second image, and determine the second transformation relationship between the first coordinate system and the third coordinate system based on the initial transformation relationship, the first image, and the second image. Further, the processor 102 may determine the positioning information of the robot based on the second transformation relationship. More descriptions regarding the determination of the positioning information of the robot may be found elsewhere in the present disclosure. See, e.g., FIG. 22 and relevant descriptions thereof.

In some embodiments, the processor 102 may further determine whether the positioning information satisfies a preset condition. The preset condition indicates that registration accuracy based on the positioning information is satisfied. For example, a registration error is less than an acceptable threshold. As another example, no abnormal conditions (e.g., facial occlusion, deformation, defects, etc.) exist under the positioning information. In some embodiments, the determined positioning information (e.g., the third transformation relationship between the second coordinate system corresponding to the robot and the third coordinate system corresponding to the medical imaging device, the registration error between the second and third coordinate systems) may be presented to the user, and the user may determine whether the positioning information satisfies the preset condition. For example, the registration error may be a comprehensive registration error or a registration error for each pixel. Based on the registration error for each pixel, a registration error heat map of the face may be generated and displayed to the user. For instance, in the display interface, the left side may display a registration effect between facial point cloud data (corresponding to the first image) and a 3D reconstructed image (reconstructed based on the second image), and the right side may display the registration error heat map. In the registration error heat map, different colors may be assigned to actual results based on the registration error, allowing the user to intuitively assess the actual registration effect based on the colors. If the registration fails, re-registration may be performed.

If the positioning information satisfies the preset condition, the processor 102 may end the process 2100. That is, the processor 102 may designate the positioning information as target positioning information. The target positioning information refers to final positioning information of the robot.

If the positioning information does not satisfy the present condition, the processor 102 may proceed to operation 2110.

In 2110, the processor 102 (e.g., the positioning module 230) may determine updated positioning information of the robot based on a fusion image, the second image, and the positioning information.

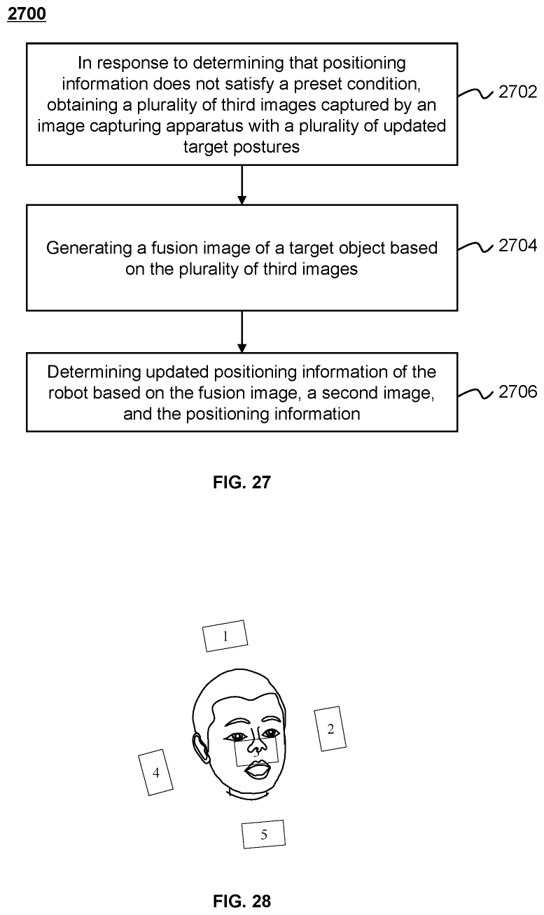

For example, the processor 102 may obtain a plurality of third images captured by the image capturing apparatus with a plurality of updated target postures, and generate the fusion image of the target object based on the plurality of third images. The processor 102 may determine the updated positioning information of the robot based on the fusion image, the second image, and the positioning information. More descriptions regarding the determination of the updated positioning information of the robot may be found elsewhere in the present disclosure. See, e.g., FIG. 27 and relevant descriptions thereof.

According to some embodiments of the present disclosure, the image capturing apparatus can be adjusted to the first target posture in the base coordinate system corresponding to the robot based on the target image. Therefore, the image capturing apparatus can be positioned to the optimal position to determine the positioning information of the robot, thereby improving the accuracy and efficiency of image data capture, and further improving the accuracy and efficiency of subsequent robot positioning and preoperative planning.

FIG. 22 is a flowchart illustrating an exemplary process 2200 for determining positioning information of a robot according to some embodiments of the present disclosure. In some embodiments, the positioning information described in operation 2108 of FIG. 21 may be determined according to the process 2200.

To accurately control operations of the robots, it is necessary to position the robots. For example, point cloud data of the head or face of a patient is obtained by an image capturing apparatus (e.g., a structured light camera), and facial reconstruction is performed on the patient's medical image data to obtain reconstructed facial point cloud data. The point cloud data of the head or face and the reconstructed facial point cloud data are registered to determine position information of the robot. However, this registration manner involves substantial computation and slow registration speed, leading to low registration efficiency. Furthermore, if the point cloud data of the head or face includes facial anomaly data, the complexity and difficulty of the registration manner can be enhanced, thereby further reducing the registration efficiency and accuracy. Therefore, it is necessary to provide an effective system and method for robot positioning. In some embodiments, the robot may be positioned by performing the following operations in the process 2200.

In 2202, the processor 102 (e.g., the positioning module 230) may determine an initial transformation relationship between a first coordinate system corresponding to an image capturing apparatus and a third coordinate system corresponding to a medical imaging device based on a first 2D reference image of a target object and a second image.

In some embodiments, the first 2D reference image may be captured by the image capturing apparatus with the first target posture. For example, the first 2D reference image may be an RGB image. The second image refers to a medical image captured using the medical imaging device (e.g., the medical imaging device 120). More descriptions regarding the first 2D reference image and the second image may be found elsewhere in the present disclosure. See, e.g., FIGS. 3, 4, and 21 and relevant descriptions thereof.

The initial transformation relationship (also referred to as a coarse registration result) refers to a coarse transformation relationship between the first coordinate system and the third coordinate system. For example, the initial transformation relationship may include a coarse registration relationship (e.g., a coarse registration matrix), a coarse registration error, or the like, or any combination thereof. The coarse registration relationship may reflect a coarse corresponding relationship and/or a coarse coordinate transformation relationship between the first coordinate system and the third coordinate system.

In some embodiments, the processor 102 may identify first feature points from the first 2D reference image, and identify second feature points from a facial reconstruction image corresponding to the second image. Further, the processor 102 may determine the initial transformation relationship by registering the first feature points and the second feature points.

A feature point refers to a unique point of the target object that characterizes its key geometric features. Exemplary feature points may include the glabella, eye corners, nose tip, mouth corners, or the like, or any combination thereof. For example, referring to FIG. 23, feature points may include outer canthus D1 and D2, inner canthus E1 and E2, nasion O, and nose tip B. In some embodiments, the number and type of the first feature points are preset.

In some embodiments, the processor 102 may automatically identify the first feature points from the first 2D reference image. For example, the processor 102 may input the first 2D reference image into a feature point identification model, and the feature point identification model may output the first feature points. The feature point identification model may be a machine learning model. For example, the feature point identification model may be obtained by training an initial feature point identification model based on a plurality of second training samples. Each of the second training samples may include a sample 2D reference image of a sample object and corresponding sample feature points labelled by a user.

In some embodiments, at least a portion of the first feature points may be labeled by a user on the first 2D reference image. For example, the processor 102 may display the first 2D reference image through a display interface of a display device, and the user may manually select the first feature points or a portion of the first feature points on the first 2D reference image. The processor 102 may determine the first feature points based on the user's marking operations on the first 2D reference image. As another example, when the user manually marks the first feature points, the processor 102 may determine a type of each marked first feature point (e.g., the right outer canthus) based on the marked first feature point and its position on the face in the first 2D reference image, thereby recording the user-marked first feature points.

In some embodiments, the processor 102 may first automatically identify the first feature points from the first 2D reference image, and display the first feature points on the first 2D reference image to the user. The user may adjust the locations of the automatically marked first feature points. This can improve the efficiency and accuracy of the marking of the first feature points.

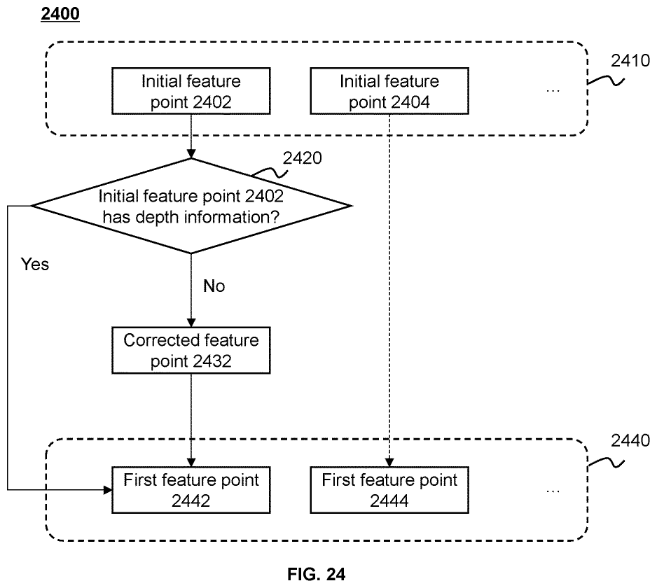

In some embodiments, the processor 102 may identify initial feature points from the first 2D reference image. For each initial feature point, the processor 102 may determine whether the initial feature point has depth information in the first image. If the initial feature point has depth information in the first image, the processor 102 may designate the initial feature point as one of the first feature points. If the initial feature point does not have depth information in the first image, the processor 102 may determine a corrected feature point as one of the first feature points based on the initial feature point and the first image. More descriptions regarding the determination of the first feature points may be found elsewhere in the present disclosure. See, e.g., FIG. 24 and relevant descriptions thereof.

In some embodiments, the processor 102 may identify the second feature points from the facial reconstruction image corresponding to the second image. For example, the processor 102 may obtain the facial reconstruction image by performing facial reconstruction based on the second image, and identify the second feature points from the facial reconstruction image. In some embodiments, the processor 102 may further perform full-head reconstruction based on the second image to obtain a full-head reconstruction image of the target object, and then extract the second feature points from the full-head reconstruction image. The second feature points may be identified in a similar manner as how the first feature points are identified, which are not repeated herein. There is a one-to-one correspondence between the first feature points and the second feature points.

In some embodiments, the processor 102 may determine the initial transformation relationship by performing registration between the first feature points and the second feature points based on a registration algorithm. The processor 102 may further determine a registration error corresponding to the initial transformation relationship. For example, the processor 102 may perform 2D-3D registration between the first feature points and the second feature points to determine the initial transformation relationship. For instance, the first feature points may correspond to first X coordinates and first Y coordinates, and the second feature points may correspond to second X coordinates, second Y coordinates, and second Z coordinates. The processor 102 may perform coarse registration based on the first X coordinates, the first Y coordinates, the second X coordinates, and the second Y coordinates to determine the initial transformation relationship.

As another example, the processor 102 may perform 3D-3D registration between the first feature points and the second feature points to determine the initial transformation relationship. For instance, the processor 102 may determine first Z coordinates based on the first image (e.g., the depth information in the first image), and perform the coarse registration based on the first X coordinates, the first Y coordinates, the first Z coordinates, the second X coordinates, the second Y coordinates, and the second Z coordinates to determine the initial transformation relationship.

In 2204, the processor 102 (e.g., the positioning module 230) may determine a second transformation relationship between the first coordinate system and the third coordinate system based on the initial transformation relationship, the first image, and the second image.

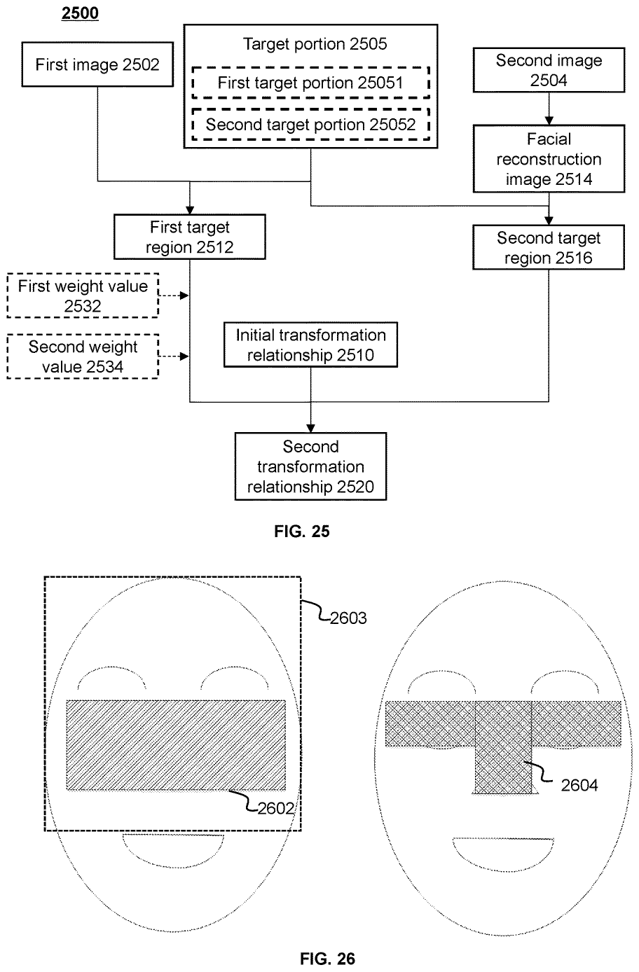

In some embodiments, the processor 102 may determine at least one first target region corresponding to at least one target portion of the target object from the first image, and determine at least one second target region corresponding to the at least one target portion from the facial reconstruction image corresponding to the second image. Further, the processor 102 may determine the second transformation relationship (also referred to as a fine registration result) by registering the at least one first target region and the at least one second target region. The initial transformation relationship may serve as an initial value for the registration (also referred to as a fine registration). More descriptions regarding the determination of the second transformation relationship may be found elsewhere in the present disclosure. See, e.g., FIG. 25 and relevant descriptions thereof.

In some embodiments, the processor 102 may determine the second transformation relationship by registering the first image and the at least one second target region corresponding to the at least one target portion from the facial reconstruction image corresponding to the second image. By registering the first image and the at least one second target region, no first target region needs to be determined, thereby reducing the complexity of the fine registration.

In some embodiments, the processor 102 may determine the second transformation relationship by registering the at least one first target region and the facial reconstruction image (or the second image). By registering the at least one first target region and the facial reconstruction image (or the second image), no second target region needs to be determined, thereby reducing the complexity of the fine registration. Furthermore, since the facial reconstruction image or the second image includes whole information of the target object, the accuracy of the fine registration can be improved.

In 2206, the processor 102 (e.g., the positioning module 230) may determine positioning information of the robot based on the second transformation relationship.

For example, the positioning information of the robot may be determined based on the second transformation relationship and a first transformation relationship. The first transformation relationship is a transformation relationship between the first coordinate system corresponding to the image capturing apparatus and the second coordinate system corresponding to the robot. More descriptions regarding the determination of the positioning information of the robot may be found elsewhere in the present disclosure. See, e.g., FIGS. 3, 5, and 21 and relevant descriptions thereof.

According to some embodiments of the present disclosure, the coarse registration can be performed on the first feature points and the second feature points to determine the initial transformation relationship, and the fine registration can be performed on the at least one first target region and the at least one second target region based on the initial transformation relationship to determine the second transformation relationship. Since the number of feature point pairs and the volume of the point cloud data within the local region are far less than the volume of overall point cloud data of the first image and the second image, the difficulty of robotic arm registration can be reduced, and the registration efficiency can be improved. Furthermore, by performing the point-to-point coarse registration and the point cloud fine registration, the registration efficiency and the registration accuracy can be improved.

FIG. 24 is a schematic diagram illustrating an exemplary process 2400 for determining first feature points according to some embodiments of the present disclosure.

As illustrated in FIG. 24, the processor 102 (e.g., the positioning module 230) may identify initial feature points 2410 from the first 2D reference image. For each initial feature point, the processor 102 may determine whether the initial feature point has depth information in the first image. The first image may be a depth image. If the initial feature point has depth information in the first image, the processor 102 may designate the initial feature point as one of first feature points 2440. If the initial feature point does not have depth information in the first image, the processor 102 may determine a corrected feature point as one of the first feature points 2440 based on the initial feature point and the first image.

Taking an initial feature point 2402 as an example, the processor 102 may determine whether the initial feature point 2402 has depth information in the first image. If the initial feature point 2402 has depth information in the first image, the processor 102 may designate the initial feature point 2402 as one of first feature points 2440 (i.e., a first feature point 2442). If the initial feature point 2402 does not have depth information in the first image, the processor 102 may determine a corrected feature point 2432 based on the initial feature point 2402 and the first image, and designate the corrected feature point 2432 as the first feature point 2442.

In some embodiments, for each initial feature point, the processor 102 may determine whether the initial feature point 2402 has the depth information in the first image by determining whether the initial feature point 2402 is located within a valid region of the first 2D reference image. The valid region refers to a pixel region with the depth information. For example, the processor 102 may align the first image and the first 2D reference image to obtain an aligned image. The processor 102 may determine, from the aligned image, first points in the first 2D reference image having point cloud holes and second points in the first 2D reference image not having point cloud holes. A first point having a point cloud hole may represent the point does not have the depth information in the first image (i.e., has a value of zero in the first image), and a second point not having the point cloud hole may represent the point has the depth information in the first image (i.e., has a value greater than zero in the first image). The processor 102 may further determine the valid region of the first 2D reference image based on the first points and/or the second points. For instance, the processor 102 may segment the first points and/or the second points from the first 2D reference image, and determine a region corresponding to the second points as the valid region of the first 2D reference image. Further, the processor 102 may determine whether the initial feature point 2402 is located within the valid region of the first 2D reference image based on a location (e.g., coordinates) of the initial feature point 2402 in the first 2D reference image.

In some embodiments, if the initial feature point 2402 does not have the depth information, the processor 102 may determine the corrected feature point 2432 by correcting the initial feature point 2402. For example, the processor 102 may determine whether points with depth information exist within a preset range around the initial feature point 2402. If one or more points with depth information exist within the preset range, one point with the depth information among them may be taken as the corrected feature point 2432. For instance, any point with the depth information within the preset range may be selected as the corrected feature point 2432. As another example, one point with the depth information nearest the initial feature point 2402 may be selected as the corrected feature point 2432. As still another example, the processor 102 may determine target depth information for the initial feature point 2402 based on the one or more points with the depth information. For instance, the processor 102 may determine an average value or a median value based on the depth information of the one or more points, and designate the average value or the median value as the depth information of the initial feature point 2402. If no point with depth information exists within the preset range, a prompt message may be output to inform the user that first feature point extraction failed. In this case, the user may be prompted to adjust a posture (e.g., a first target posture) of the image capturing apparatus to recapture the target object and obtain a new first image and a new first 2D reference image, so as to determine new first feature points from the new first 2D reference image.

As another example, preset depth information may be determined in advance, and the processor 102 may retrieve the preset depth information to assign to the initial feature point 2402. For instance, the processor 102 may divide the face of the target object into a plurality of sub-regions, and determine the preset depth information for each of the plurality of sub-regions. The processor 102 may determine a target sub-region where the initial feature point 2402 is located, and assign the preset depth information corresponding to the target sub-region to the initial feature point 2402.

A first feature point 2444 may be determined based on the initial feature point 2404 in a similar manner as how the first feature point 2442 is determined.

According to some embodiments of the present disclosure, by determining whether each initial feature point has depth information (i.e., verifying the validity of each initial feature point) and correcting those that lack such information, it is ensured that every first feature point has corresponding depth information. This facilitates subsequent point-to-point coarse registration based on the first feature points and corresponding second feature points, thereby reducing registration error and enhancing registration precision.

FIG. 25 is a schematic diagram illustrating an exemplary process 2500 for determining a second transformation relationship according to some embodiments of the present disclosure.

As illustrated in FIG. 25, the processor 102 (e.g., the positioning module 230) may determine at least one first target region 2512 corresponding to at least one target portion 2505 of a target object from a first image 2502. The processor 102 may generate a facial reconstruction image 2514 corresponding to a second image 2504, and determine at least one second target region 2516 corresponding to the at least one target portion 2505 from the facial reconstruction image 2514. The processor 102 may determine a second transformation relationship 2520 by registering the at least one first target region 2512 and the at least one second target region 2516.

The at least one target portion 2505 refers to a local region of the target object. Taking the face as an example, the at least one target portion 2505 may be a local facial region, such as a bony region of the face, a large facial region, etc. For example, the at least one target portion 2505 may include a forehead region, a cheek region, a region between the forehead and the nose tip, a region between the nose tip and the chin, a region between eyes and lips, or the like, or any combination thereof. In some embodiments, the at least one target portion 2505 may be less affected by changes in facial expressions than other portions of the target object. For example, referring to FIG. 26, the target object is the face, and the at least one target portion of the target object may correspond to a region of the face enclosed by the box 2602 or the box 2603. As another example, the at least one target portion 2505 incudes the whole face.

In some embodiments, the processor 102 may determine the at least one first target region 2512 (also referred to the first point cloud data) corresponding to the at least one target portion 2505 of the target object from the first image 2502. For example, the processor 102 may obtain a regional image corresponding to the at least one target portion 2505 by cropping the first image 2502, and obtain the at least one first target region 2512 corresponding to the at least one target portion 2505 by performing point cloud extraction on the regional image. In some embodiments, the at least one first target region 2512 is determined based on the first 2D reference image. More descriptions regarding the determination of the at least one first target region may be found elsewhere in the present disclosure. See, e.g., FIGS. 3 and 4 and relevant descriptions thereof.

In some embodiments, the processor 102 may determine the at least one second target region 2504 (also referred to second point cloud data) corresponding to the at least one target portion 2505 from the facial reconstruction image 2514 (or a full-head reconstruction image) corresponding to the second image 2504. The at least one second target region 2516 may be determined in a similar manner as how the at least one first target region 2512 is determined, which is not repeated herein.

In some embodiments, the processor 102 may determine the second transformation relationship 2520 by registering the at least one first target region 2512 and the at least one second target region 2516 based on an initial transformation relationship 2510. For example, the processor 102 may perform fine registration on the at least one first target region 2512 and the at least one second target region 2516 based on the initial transformation relationship 2510 to determine the second transformation relationship 2520. For instance, the initial transformation relationship 2510 may serve as an initial value for the fine registration, and iterative operations are performed to update the initial value based on the at least one first target region 2512 and the at least one second target region 2516 until a preset iteration stopping condition is met. The updated initial value after the preset iteration stopping condition is met is designated as the second transformation relationship 2520. In some embodiments, the preset iteration stopping condition may include that iteration counts reaches a preset number of iterations, meeting registration accuracy requirements, or the like, or any combination thereof. More descriptions regarding the determination of the initial transformation relationship may be found elsewhere in the present disclosure. See, e.g., FIG. 22 and relevant descriptions thereof.

In some embodiments, the at least one target portion 2505 may include a first target portion 25051 and a second target portion 25052, and the first target portion 25051 may be less affected by changes in facial expressions than the second target portion 25052. For example, the first target portion 25051 may be a region where the degree of facial deformation is less than or equal to a preset deformation threshold. The second target portion 25052 may be a region where the degree of facial deformation is greater than the preset deformation threshold. For instance, the first target portion 25051 may be the bony region (also referred to as a static facial region), and the second target portion 25052 may be a non-bony region. As another example, the at least one target portion 2505 may be an entire area from the forehead to the nose tip on the face. The first target portion 25051 may be a less deformable region including the forehead and nose tip, and the second target portion 25052 may be a deformable region including the eyes and cheeks.

In some embodiments, the fine registration may be performed further based on a first weight value 2532 corresponding to the first target portion 25051 and a second weight value 2534 corresponding to the second target portion 25052, and the first weight value 2532 may be greater than the second weight value 2534.

In some embodiments, the processor 102 may determine the first weight value 2532 corresponding to the first target portion 25051 and the second weight value 2534 corresponding to the second target portion 25052, and perform the fine registration based on the first weight value 2532, the second weight value 2534, the at least one first target region 2512, the at least one second target region 2516, and the initial transformation relationship 2510.

In some embodiments, the processor 102 may assign pre-set fixed weights to the first target portion 25051 and the second target portion 25052. Alternatively, the first weight value 2532 corresponding to the first target portion 25051 and the second weight value 2534 corresponding to the second target portion 25052 may be determined based on a deformation degree of the second target portion 25052. For example, the higher the deformation degree of the second target portion 25052, the smaller the corresponding second weight value 2534, and accordingly the larger the first weight value 2532. That is, when severe deformation occurs in the second target portion 25052, the importance of the second target portion 25052 in the fine registration may be reduced to avoid significant errors in the second transformation relationship 2520 caused by the severely deformed second target portion 25052.

Merely by way of example, a deformation degree of a portion may be classified into multiple levels from high to low or low to high (e.g., severe deformation, moderate deformation, mild deformation). Different weight values may be set for different levels of deformation, thereby establishing a corresponding relationship between different deformation levels and their corresponding weight values. The regional image corresponding to the at least one target portion 2505 may be cropped from the first image 2502, and then a sub-regional image (also referred to as second sub-point cloud) corresponding to the at least one second target portion 25052 may be cropped from the regional image. The deformation degree of the at least one second target portion 25052 in the sub-regional image may be determined, and the second weight value 2534 corresponding to the second target portion 25052 may be determined based on the deformation degree of the second target portion 25052 in the sub-regional image and the corresponding relationship. Accordingly, the first weight value 2532 corresponding to the first target portion 25051 may be determined based on the second weight value 2534 corresponding to the second target portion 25052, wherein a sum of the first weight value 2532 and the second weight value 2534 may be 1.

In some embodiments, the first target region 2512 (also referred to as first point cloud data) may include first sub-point cloud corresponding to the at least one first target portion 25051 and the second sub-point cloud corresponding to the at least one second target portion 25052. The second target region 2516 (also referred to as second point cloud data) may include third sub-point cloud corresponding to the at least one first target portion 25051 and fourth sub-point cloud corresponding to the at least one second target portion 25052. The processor 102 may perform the fine registration based on the first weight value 2532 corresponding to the first target portion 25051, the second weight value 2534 corresponding to the second target portion 25052, the first to fourth sub-point cloud data, and the initial transformation relationship 2510, so as to determine the second transformation relationship 2520.

For example, the fine registration includes a plurality of iterations. In each iteration of the fine registration process, a current registration matrix between the at least one first target region and the at least one second target region is obtained. Each point in the first sub-point cloud and the second sub-point cloud is transformed using this matrix. Subsequently, for each transformed point, the nearest neighboring point is identified within the third sub-point cloud and the fourth sub-point cloud. An objective function, which quantifies the registration error, is then calculated based on the distances between each transformed point and its corresponding nearest neighbor, weighted by the respective first or second weight. The registration matrix is then updated according to the value of this objective function, and the fine registration process proceeds to the next iteration. The iterations are terminated when a preset iteration stopping condition is satisfied.

In some embodiments, each point in the first target region (e.g., the bony region) is assigned a corresponding first weight, where points with more pronounced bony characteristics are given larger weights. Similarly, each point in the second target region (e.g., the non-bony region) is assigned a corresponding second weight, where points with more pronounced non-bony characteristics are given smaller weights. The first weights are within a first range, and the second weights are within a second range, with the values in the first range being greater than those in the second range.

In some embodiments, the processor 102 may determine the second transformation relationship 2520 by registering the at least one first target region 2512 and the facial reconstruction image 2514 (or the second image 2504). The fine registration on the at least one first target region 2512 and the facial reconstruction image 2514 (or the second image 2504) may be performed in a similar manner as how the fine registration on the at least one first target region 2512 and the at least one second target region 2516 is performed as described above, which is not repeated herein.

By setting the first weight value greater than the second weight value, the first target portion can have a larger influence on the fine registration than the second target portion. Since the first target portion is less affected by changes in facial expressions than the second target portion, registration errors caused by facial deformations in the second target portion during the fine registration can be avoided, thereby enhancing the accuracy of the fine registration.

FIG. 27 is a flowchart illustrating an exemplary process 2700 for determining updated positioning information of a robot according to some embodiments of the present disclosure. In some embodiments, the updated positioning information described in operation 2110 of FIG. 21 may be determined according to the process 2700.

In some embodiments, after the positioning information is determined, the processor 102 may determine whether the positioning information satisfies a preset condition. If the positioning information does not satisfy the present condition, the processor 102 may the process 2700.

In 2702, the processor 102 (e.g., the positioning module 230) may obtain a plurality of third images captured by an image capturing apparatus with a plurality of updated target postures.

In some embodiments, the processor 102 may determine the plurality of updated target postures of the image capturing apparatus at a base coordinate system based on the second image and the positioning information, and cause the robot to move the image capturing apparatus to the plurality of updated target postures, respectively, to obtain the plurality of third images. The count of the updated target postures may be two or more than two.

For example, the processor 102 may perform full-head reconstruction based on the second image to obtain a full-head reconstruction image and/or head information. The head information may include face orientation, bounding sphere parameters (e.g., center and radius of the bounding sphere for the face), etc. More descriptions regarding the determination of the full-head reconstruction image may be found elsewhere in the present disclosure. See, e.g., FIG. 22 and relevant descriptions thereof. The processor 102 may determine the plurality of updated target postures around the target object (e.g., the head) based on the full-head reconstruction image and/or the head information and the positioning information determined in operation 2108. For example, the processor 102 may determine initial updated postures in the third coordinate system corresponding to the medical imaging device based on the full-head reconstruction image, and then transform the initial updated postures into the updated target postures in the base coordinate system based on the positioning information.

Merely by way of example, as illustrated in FIG. 28, the plurality of updated target postures may include a posture 3 directly above the face, a posture 4 with a certain tilt angle to the right of the face, a posture 2 with a certain tilt angle to the left of the face, a posture 1 with a certain tilt angle above the head, and a posture 5 with a certain tilt angle below the chin, etc.

In some embodiments, the processor 102 may control the robot (e.g., a robotic arm of the robot) to move the image capturing apparatus sequentially to each of the plurality of updated target postures. Alternatively, a user may manually move the image capturing apparatus sequentially to each of the plurality of updated target postures. In some embodiments, at each updated target posture, the image capturing apparatus may be caused to capture a corresponding third image (e.g., a depth image).

In 2704, the processor 102 (e.g., the positioning module 230) may generate a fusion image of the target object based on the plurality of third images.

For example, the processor 102 may fuse the plurality of third images (e.g., through stitching, downsampling, and cropping) to generate the fusion image of the target object. As another example, the processor 102 may register the plurality of third images and fuse the registered third images to generate the fusion image of the target object.

In 2706, the processor 102 (e.g., the positioning module 230) may determine updated positioning information of the robot based on the fusion image and the second image.

For example, the processor 102 may perform a second fine registration based on the fusion image, the second image, and the positioning information. For instance, the positioning information may serve as a second initial value for the second fine registration, and iterative operations are performed based on the second initial value, the fusion image, the second image until a second preset iteration stopping condition is met, thereby determining the updated positioning information. The updated positioning information may be determined in a similar manner as how the positioning information is determined, which is not repeated herein.

According to some embodiments of the present disclosure, for cases with facial anomalies or when initial registration accuracy is insufficient, multi-posture image capture and fusion can be leveraged to provide a more comprehensive image (i.e., the fusion image) for the second fine registration. This generates more accurate updated positioning information, enhancing the precision and reliability of the robot positioning.

FIG. 29 is a flowchart illustrating an exemplary process 2900 for adjusting an image capturing apparatus according to some embodiments of the present disclosure. In some embodiments, the semi-automatic adjustment of the image capturing apparatus described in operation 2104 of FIG. 21 may be determined according to the process 2900.

In 2902, the processor 102 (e.g., the posture determination module 930) may control, based on the target image, a robot to adjust an image capturing apparatus to a target shooting angle or cause a display device to display, based on the target image, guidance information for guiding a user to adjust the image capturing apparatus to the target shooting angle.

The target shooting angle may be an angle directly facing the target object (e.g., a patient's face).

For example, the processor 102 may control the robot to adjust the image capturing apparatus to the target shooting angle based on the target image. As another example, the processor 102 may control the robot to adjust the image capturing apparatus to the target shooting angle based on the first target posture in the base coordinate system. More descriptions regarding the adjusting the image capturing apparatus to the target shooting angle may be found elsewhere in the present disclosure. See, e.g., FIG. 10 and relevant descriptions thereof.

As another example, the processor 102 may cause the display device to display the guidance information. For instance, a display interface of the display device may be used to display the guidance information.

The guidance information may be used to guide the user to manually adjust the image capturing apparatus to the target shooting angle. In some embodiments, the guidance information may include a reference contour corresponding to the head of the target object, a current 2D reference image, offset guidance information determined based on a current contour of the head in the current 2D reference image and the reference contour, or the like, or any combination thereof. The reference contour is a static contour, such as a facial outline viewed from the front. It should be noted that the reference contour is only used to indicate the contour shape of the head, not the specific head contour of the target object.

The current 2D reference image may be the current 2D image captured by the image capturing apparatus. For example, the current 2D reference image is a second 2D reference image or a fourth 2D reference image. The second 2D reference image (included in the target image) may be captured by the image capturing apparatus with the initial posture (i.e., before the posture of the image capturing apparatus is adjusted). The fourth 2D reference image may be captured by the image capturing apparatus during the adjustment of the image capturing apparatus to the target shooting angle. The current 2D reference image is updated in real-time during the posture adjustment process of the image capturing apparatus.

In some embodiments, the current 2D reference image and the reference contour may be displayed in the display interface to display a relative positional relationship between a current posture of the target object and the reference contour. The relative positional relationship between the current contour of the head in the current 2D reference image and the reference contour may be used to guide the user to manually adjust the image capturing apparatus to the target shooting angle, for example, align the current posture of the target object and the reference contour. The alignment refers to that organs (e.g., the forehead, ears, chin, etc.) of the target object are roughly aligned with the reference contour.

For example, referring to FIG. 30, FIG. 30 is a schematic diagram illustrating an exemplary display interface 3000 according to some embodiments of the present disclosure. As illustrated in FIG. 30, the display interface 3000 may display a reference contour 3002 and a current contour of the head in a current 2D reference image 3004 of the target object. According to a relative positional relationship in the display interface 3000, a user may manually adjust the image capturing apparatus to the target shooting angle, so as to align the current contour of the head in the current 2D reference image 3004 and the reference contour 3002.

The offset guidance information may include an offset direction, an offset distance, an offset angle, or the like, or any combination thereof. In some embodiments, the processor 102 may determine the offset guidance information by performing offset analysis on the reference contour and the current contour in the current 2D reference image. For example, when the head is offset to the left relative to the reference contour, the offset guidance information may guide the user to move right. That is, the offset direction in the guidance information is opposite to the offset direction of the target object relative to the reference contour.

In some embodiments, the guidance information further includes feature points identified in the current 2D reference image (e.g., the right outer corner of the eye, right inner corner of the eye, left outer corner of the eye, left inner corner of the eye, tip of the nose).

Displaying the offset guidance information on the display interface can help the user quickly move the image capturing apparatus, thereby quickly and accurately aligning with the reference contour on the display interface and moving the image capturing apparatus to the first target posture. This not only reduces the difficulty of user operation but also improves the accuracy of manual operation, avoiding cumbersome re-adjustment of the image capturing apparatus later due to inaccurate capture posture, thereby improving the efficiency of the manual operation.

As the user manually moves the image capturing apparatus (e.g., by moving the robotic arm of the robot where the image capturing apparatus is mounted), the fourth 2D reference image(s) and the offset guidance information may be updated in real-time, allowing the user to accurately align the target object's head with the reference contour. The process may continue until the target object's head in the fourth 2D reference image matches the reference contour or until the user provides a confirmation input, indicating the target shooting angle has been reached.

In 2904, the processor 102 (e.g., the posture determination module 930) may obtain a candidate image of the target object captured by the image capturing apparatus from the target shooting angle.

Once the image capturing apparatus has been positioned at the target shooting angle (either automatically or through user operation), the processor 102 may obtain the candidate image of the target object captured from the target shooting angle. The candidate image may include depth information. For example, the candidate image may be a depth image. As another example, the candidate image may be an RGB image. The candidate image may be captured in a similar manner as how the target image is captured as described in operation 2102, which is not repeated herein.

In some embodiments, the user may control the image capturing apparatus to capture the candidate image after the user confirms that the image capturing apparatus is adjusted to the target shooting angle facing the target object. In some embodiments, the processor 102 may automatically control the image capturing apparatus to capture the candidate image upon determining that the current shooting angle is the target shooting angle (e.g., when the head of the target object is positioned within the reference contour and aligned with the structure of the reference contour). Alternatively, the processor 102 may designate the currently captured image as the candidate image upon determining that the current shooting angle is the target shooting angle.

In 2906, the processor 102 (e.g., the posture determination module 930) may control, based on the candidate image, the robot to adjust the image capturing apparatus to a target shooting distance from the target object.

The processor 102 may determine whether a current shooting distance of the image capturing apparatus satisfies shooting requirements. For example, the processor 102 may determine whether the current shooting distance of the image capturing apparatus satisfies the shooting requirements based on a third 2D reference image captured by the image capturing apparatus from the target shooting angle and the candidate image. If the current shooting distance of the image capturing apparatus satisfies the shooting requirements, the processor 102 may determine the current shooting distance as the target shooting distance. If the current shooting distance of the image capturing apparatus does not satisfy the shooting requirements, the processor 102 may determine the target shooting distance based on the candidate image and cause the robot to adjust the image capturing apparatus to the target shooting distance. The third 2D reference image may be an RGB image captured simultaneously with the candidate image from the target shooting angle.

In some embodiments, when the candidate image is the RGB image, the processor 102 may determine whether the current shooting distance of the image capturing apparatus satisfies the shooting requirements based on the RGB image. That is, the candidate image may be also referred to as the third 2D reference image.

The shooting requirements may include that a depth distance between the image capturing apparatus and the target object is within a preset distance threshold. The depth distance refers to a distance along a depth direction, and the depth direction refers to a vertical direction between the image capturing apparatus and the target object, i.e., a projection direction of the image capturing apparatus onto the target object. For example, when the target object is in a supine position, the depth direction is a height direction of the image capturing apparatus relative to the target object. In some embodiments, the preset distance threshold may be manually set by the user or determined according to system settings.

In some embodiments, the processor 102 may determine the depth distance between the image capturing apparatus and the target object based on the candidate image. In some embodiments, the depth distance between the image capturing apparatus and the target object may be represented by a depth distance between the image capturing apparatus and the nose tip of the target object.

In some embodiments, the processor 102 may determine third feature points in the third 2D reference image captured, and determine whether the third feature points have depth information in the candidate image. If one or more of the third feature points do not have depth information in the candidate image, the processor 102 may determine that the current shooting distance does not satisfy the shooting requirements. If each of the third feature points has the depth information in the candidate image, the processor 102 may determine that the current shooting distance satisfies the shooting requirements. The third feature points may be similar to the first feature points, and be determined in the third 2D reference image in a similar manner as how the first feature points are determined in the first 2D reference image.

In some embodiments, the processor 102 may further determine the target shooting distance based on the candidate image. For example, the processor 102 may determine the target shooting distance based on the analysis of the candidate image (e.g., the pattern or location of missing depth information). The system then generates movement control information for the depth direction (e.g., moving the apparatus closer to or farther from the target object).

In some embodiments, the processor 102 may iteratively perform the adjustment (e.g., move the image capturing apparatus by a preset step) until the shooting requirements are satisfied. In some embodiments, if after a preset number of adjustments or a preset duration, the shooting requirements are still not satisfied, it can be determined that the automatic adjustment has failed. At this point, the user can be prompted to manually adjust the height of the image capturing apparatus to meet the shooting requirements. In some embodiments, the user may input an instruction to trigger the adjustment to the target shooting distance via the display interface or an adjustment component on the robotic arm (e.g., a foot pedal). According to some embodiments of the present disclosure, the semi-automatic adjustment manner first ensures the image capturing apparatus is correctly aligned facing the target (either automatically or via user guidance), and then automatically fine-tunes the shooting distance to an optimal value (i.e., the target shooting distance) based on depth information analysis. This two-stage approach enhances user efficiency, compensates for potential manual positioning errors, and ensures the subsequent images used for registration contain the necessary depth data, thereby improving overall registration reliability and efficiency.

FIG. 31 is a flowchart illustrating an exemplary process 3100 for adjusting an image capturing apparatus to a first target posture according to some embodiments of the present disclosure. In some embodiments, the adjusting the image capturing apparatus to the first target posture described in operation 2104 of FIG. 21 may be determined according to the process 3100.

In 3102, the processor 102 (e.g., the posture determination module 930) may perform target recognition on an environmental image to determine whether a target object exists in the environmental image.

The environmental image refers to an image of the environment where the target object is located. In some embodiments, the environmental image may be captured by an image capturing apparatus mounted on a robot with an initial posture. More descriptions regarding the environmental image may be found elsewhere in the present disclosure. See, e.g., FIG. 21 and relevant descriptions thereof.

In some embodiments, the image of the target object and the environmental image may be captured by the same image capturing apparatus. For example, the processor 102 may cause the image capturing apparatus to capture the environmental image before capturing the image of the target object (e.g., the first image as described in connection with FIGS. 3 and 21).

In some embodiments, the image capturing apparatus may include a first image capturing apparatus and a second image capturing apparatus different from the first image capturing apparatus. The image of the target object may be captured by the first image capturing apparatus, and the environmental image may be captured by the second image capturing apparatus. In some embodiments, the first and second image capturing apparatus may be mounted in different positions of the robot. For example, the first image capturing apparatus is mounted on the end of the robotic arm, while the second image capturing apparatus is mounted on the middle portion of the robotic arm. Merely by way of example, the robotic arm may be a six-axis robotic arm. The second image capturing apparatus is installed near the fourth axis of the robotic arm, for example, on the surface of the connecting link between the fourth axis and the fifth axis. The second image capturing apparatus may be located near the sixth axis of the robotic arm, for example, at the distal end of the robotic arm.

In some embodiments, the processor 102 (e.g., the posture determination module 930) may perform target recognition on the environmental image to determine whether the target object exists in the environmental image. If the target object exists in the environmental image, the processor 102 may proceed to operation 3104. If the target object does not exist in the environmental image, the processor 102 may proceed to operation 3106. For example, the processor 102 may perform the target recognition using a target recognition algorithm (e.g., a pre-trained facial recognition model).

In 3104, the processor 102 (e.g., the posture determination module 930) may determine a first target posture based on the environmental image and cause the robot to adjust the image capturing apparatus to the first target posture.

The first target posture may be determined based on the environmental image including the target object in a similar manner as how the first target posture may be determined based on the target image as described in FIG. 21.

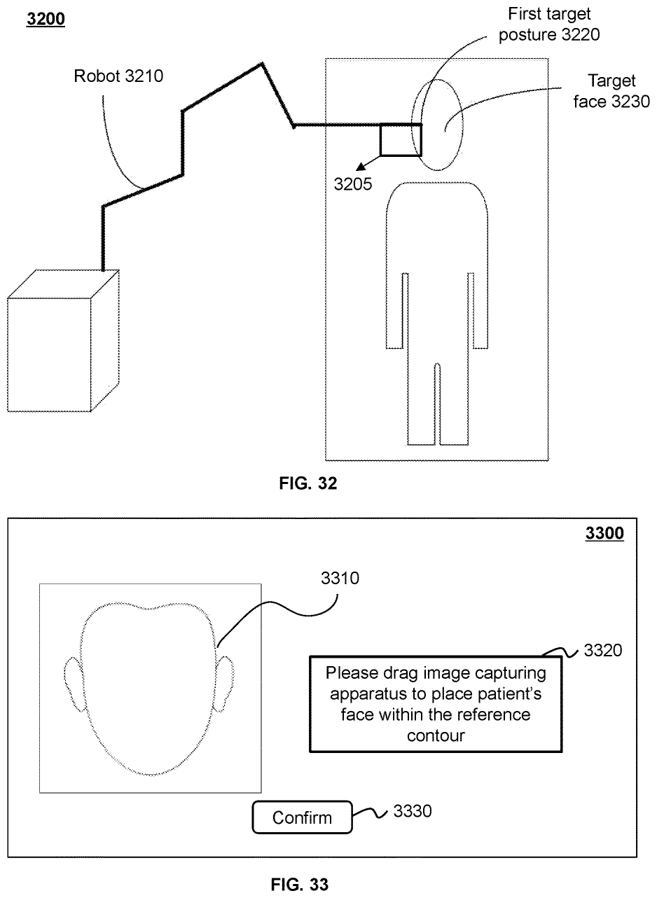

Merely by way of example, as illustrated in FIG. 32, an image capturing apparatus 3205 is mounted on a robot 3210. When a target object exists in an environmental image captured by the image capturing apparatus 3205 at an initial posture, the image capturing apparatus 3205 may be caused to move to a first target posture 3220 to capture a target face 3230 of the target object.

In 3106, the processor 102 (e.g., the posture determination module 930) may guide a user to adjust the image capturing apparatus to the first target posture.

For example, the processor 102 may cause a display device to display guidance information for guiding the user to adjust the image capturing apparatus to the first target posture. Merely by way of example, as illustrated in FIG. 33, a display interface 3300 may display guidance information (e.g., a facial reference contour 3310 and a guidance message 3320 such as “Please drag image capturing apparatus to place patient's face within the reference contour”) to guide the user to adjust the image capturing apparatus to the first target posture. The display interface 3300 may include a confirm button 3330. When the user determines that the patient's face is within the reference contour, the user may click the confirm button 3330. As another example, when the user adjusts the image capturing apparatus, the processor 102 may automatically determine whether the current posture is the first target posture based on the latest image captured by the image capturing apparatus. For example, if the whole face of the target object clearly appears in the latest image, the processor 102 may determine that the current posture is the first target posture. As another example, if the whole face of the target object clearly appears in the latest image and the facial area in the latest image exceeds a preset area threshold, the processor 102 may determine that the current posture is the first target posture.

As another example, the processor 102 may cause the display device to display the guidance information for guiding the user to adjust the image capturing apparatus to the target shooting angle, and the processor 102 may control the robot to adjust the image capturing apparatus to a target shooting distance from the target object. More descriptions regarding the manual manner and the semi-automatic manner may be found elsewhere in the present disclosure. See, e.g., FIGS. 21 and 29, and relevant descriptions thereof.

According to some embodiments of the present disclosure, this dual-path approach based on environmental image recognition ensures robust operation: fully automatic positioning is used when the target object is readily identifiable, maximizing efficiency; and user-guided positioning is seamlessly activated when automatic recognition fails, thereby ensuring the process can continue without manual re-initialization and enhancing the overall reliability and usability of the robot control system.

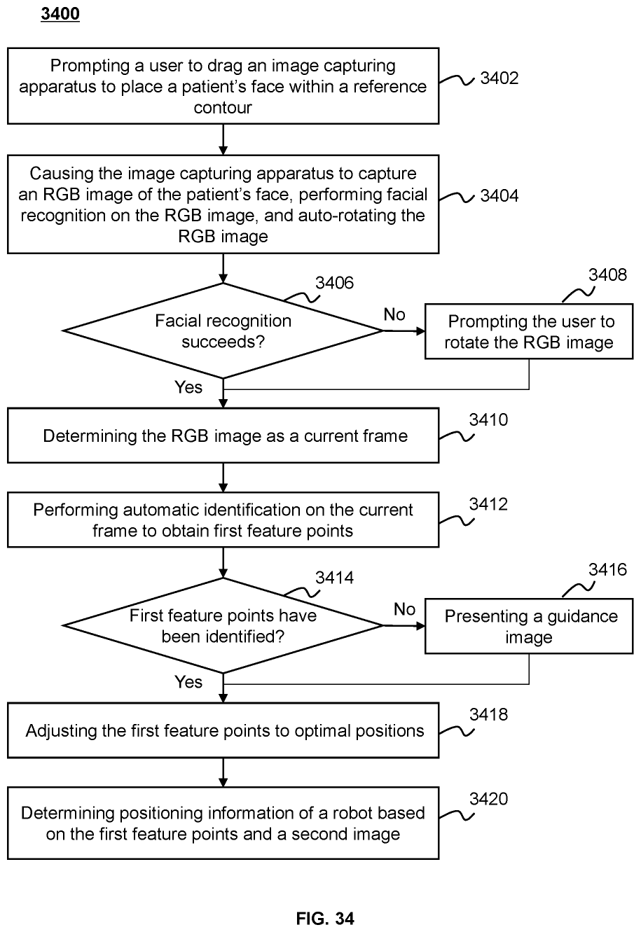

FIG. 34 is a flowchart illustrating an exemplary semi-automatic process 3400 for robot positioning according to some embodiments of the present disclosure.

When an automatic process for robot positioning fails, the processor 102 may proceed to a semi-automatic process 3400 for robot positioning.

As illustrated in FIG. 34, in 3402, the processor 102 may prompt a user to drag an image capturing apparatus to place a patient's face within a reference contour. Accordingly, the user may manually drag a robotic arm of a robot where the image capturing apparatus is mounted to position the image capturing apparatus directly above the patient's face.

In 3404, the processor 102 may cause the image capturing apparatus to capture an RGB image of the patient's face, perform facial recognition on the RGB image, and auto-rotate the RGB image.

In 3406, the processor 102 may determine whether the facial recognition succeeds. If the facial recognition fails, the processor 102 may proceed to operation 3408. If the facial recognition succeeds, the processor 102 may proceed to operation 3410.

In 3408, the processor 102 may prompt the user to rotate the RGB image. After the user rotates the RGB image, the processor 102 may proceed to operation 3410.

In 3410, the processor 102 may determine the RGB image as a current frame (also referred to as the first 2D reference image).

In 3412, the processor 102 may perform automatic identification on the current frame to obtain first feature points.

In 3414, the processor 102 may determine whether the first feature points have been identified. If the first feature points have not been identified, the processor 102 may proceed to operation 3416. If the first feature points have been obtained, the processor 102 may proceed to operation 3418.

In 3416, the processor 102 may present a guidance image for guiding the user to label the first feature points on the RGB image. After the user manually labels the first feature points on the RGB image, the processor 102 may proceed to operation 3418.

In 3418, the processor 102 may adjust the first feature points to optimal positions.

In 3420, the processor 102 may determine positioning information of the robot based on the first feature points and a second image. For example, the processor 102 may determine at least one reference region corresponding to at least one target portion from the current frame (i.e., the first 2D reference image), determine at least one target region from a first image based on the at least one reference region, and then determine positioning information based on the at least one target region and the second image.

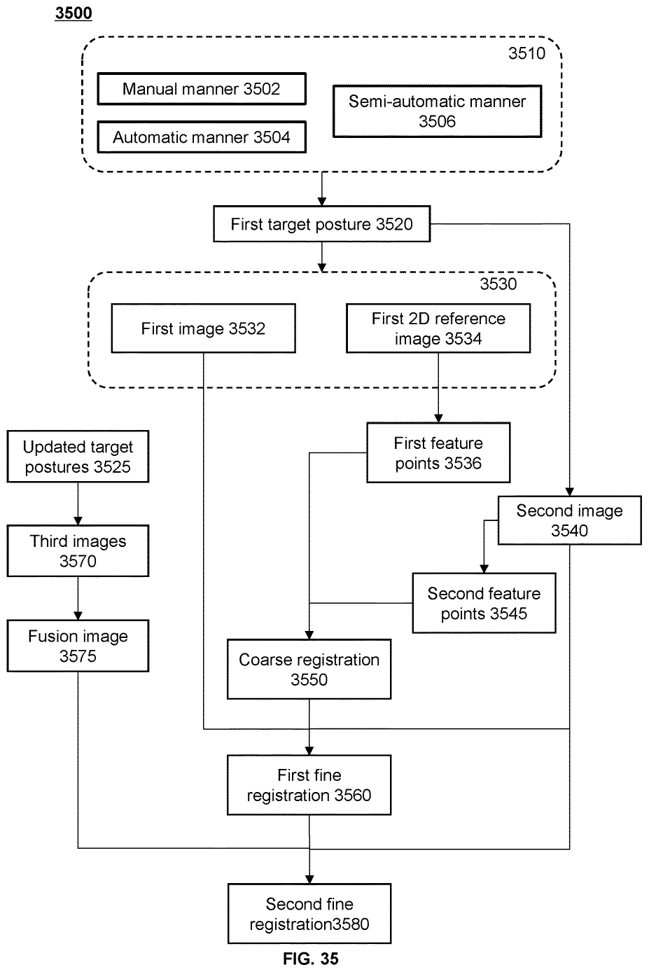

FIG. 35 is a flowchart illustrating an exemplary process 3500 for robot positioning according to some embodiments of the present disclosure.

As illustrated in FIG. 35, an image capturing apparatus may be adjusted to a first target posture 3520 using an adjustment manner 3510. The adjustment manner 3510 may include a manual manner 3502, an automatic manner 3504, and a semi-automatic manner 3506.

After the image capturing apparatus is adjusted to the first target posture 3520, the processor 102 may obtain image data 3530 captured by the image capturing apparatus and a second image 3540 of the target object. The image data 3530 may include a first image 3532 and a first 2D reference image 3534.

The processor 102 may identify first feature points 3536 from the first 2D reference image 3534, and identify second feature points 3545 based on the second image 3540. The processor 102 may perform a coarse registration 3550 on the first feature points 3536 and the second feature points 3545 to obtain a coarse registration result (i.e., an initial transformation relationship). The processor 102 may perform a first fine registration 3560 based on the first image 3532, the second image 3540, and the coarse registration result to obtain a first fine registration result (i.e., a second transformation relationship), thereby determining position information of a robot.

In some embodiments, the processor 102 may determine whether the position information satisfies a preset condition. If the position information satisfies the preset condition, the processor 102 may end the process 3500.

If the position information does not satisfy the preset condition, the processor 102 may obtain third images 3560 captured by the image capturing apparatus with updated target postures 3570, and generate a fusion image 3575 of the target object based on the third images 3570. The processor 102 may perform a second fine registration 3580 based on the fusion image 3575, the second image 3540, and the position information to obtain a second fine registration result (i.e., updated position information of the robot).

Merely by way of example, taking the image capturing apparatus as a structured light camera as an example, a complete registration process is provided. The complete registration process may include following operations.

In operation 1, upon entering the registration process, full-head reconstruction is performed based on a target object's medical image (e.g., a CT image) to obtain a full-head reconstruction image. At the same time, a user freely drags a robot (e.g., a robotic arm of the robot) and/or an image capturing apparatus to align the target object's face according to a reference contour (also referred to as a recommendation box) displayed on a display interface. A face recognition algorithm is retrieved in real-time to automatically extract first feature points of the target object's face and highlight the first feature points.

By highlighting the first feature points for display, the user can more clearly determine whether the first feature points extracted by the face recognition algorithm are indeed at the corresponding positions on the face, e.g., whether an extracted nose tip feature point is at the nose tip. Highlighting assists the user in understanding the face recognition algorithm's recognition accuracy, and the user can also visually see the recognition accuracy through the display interface.

In operation 2, during the movement of the robot and/or the image capturing apparatus, if the face's position is offset relative to the reference contour, offset guidance information may be displayed in arrow form to prompt the user in which direction to drag the image capturing apparatus to adjust the face. If the user is satisfied with the face position, the user may click the “Confirm” button on the display interface. A current frame image at confirmation is captured, and the face in the current frame image is recognized. If face recognition is successful, pixel coordinates of each first feature point on the face in the first 2D reference image are obtained, and an optimal position (i.e., the first target posture) of the image capturing apparatus may be determined. At this point, the user may step on the robotic arm's control pedal to trigger or initiate automatic control of the robotic arm. The image capturing apparatus may automatically adjust to the first target posture. If face recognition fails, the process proceeds to operation 4 with a bubble prompt for the user to perform manual point marking.

Alternatively, the image capturing apparatus may be adjusted to the first target posture in an automatic manner or a semi-automatic manner.

In operation 3, after the image capturing apparatus is adjusted to the first target posture, a target image (including an RGB image (i.e., the first 2D reference image) and a depth image (i.e., the first image)) may be captured. An aligned RGB image may be generated by aligning the RGB image and the depth image. From the aligned RGB image, pixels in the first image having point cloud holes may be determined. The pixels having the point cloud holes (also referred to as point cloud hole regions) may be marked with a different color. Non-marked regions are non-point cloud hole regions, i.e., valid regions with depth information. Facial feature point recognition may be performed on the RGB image. If the detection succeeds, multiple first feature points may be extracted from the RGB image. If the detection fails, the process may proceed to operation 4 with the bubble prompt for manual point marking. In some embodiments, the automatically extracted first feature points from the RGB image may be transformed into the aligned RGB image. If one first feature point is determined to lack depth information in the aligned RGB image (i.e., is within the point cloud hole regions), position correction may be automatically performed on the first feature point to move the first feature point to the non-point cloud hole regions (i.e., the valid region).

If the detection is successful, the process may proceed to a feature point extraction interface. The left side (CT window) of the feature point extraction interface displays CT image data, and the right side (RGB window) of the feature point extraction interface displays RGB image data. The first feature points may be extracted in the RGB window, and second feature points may be extracted in the CT window.

In operation 4, automatic extraction, manual extraction, or semi-automatic extraction (e.g., automatic extraction followed by manual adjustment) may be performed for the feature point extraction. Based on this, the valid first feature points and the corrected first feature points may be labelled in the aligned RGB image. Additionally, the second feature points may be extracted from the full-head reconstruction image and each second feature point may be labelled in the full-head reconstruction image for display. For example, the feature point extraction interface can simultaneously display the aligned RGB image labelled with the first feature points and the full-head reconstruction image labelled with the second feature points.

In the feature point extraction interface, the user may check the first feature points and/or the second feature points. For example, if the user finds the RGB image or the extraction effect of the first feature points from the RGB image unsatisfactory, the user may return to the previous step to adjust the image capturing apparatus and recapture the target image. If the user finds the automatic extraction positions of the second feature points in the CT image or the first feature points from the RGB image inaccurate or incomplete, the user may manually add feature points, drag the feature points to the best position, or delete the current feature point and re-mark a feature point manually.

In operation 5, in the feature point extraction interface, automatic validity detection and/or position correction of the feature points may be performed. If the position of one feature point is unreasonable, post-processing may be performed on the feature point to move the feature point to a reasonable position. The unreasonable position may include that the first feature point is not marked on the face or marked in the point cloud hole region of the RGB image but with the valid region nearby. In such cases, the position correction may be performed on the first feature point to move the first feature point to the nearest pixel with depth information.

It should be noted that whether automatically extracted or manually adjusted by the user, the second feature points in the CT image and the first feature points in the RGB image must be a one-to-one correspondence.

In operation 6, after the user confirms that the feature points in both the CT image and the RGB image are fully extracted and correctly positioned, clicking confirm proceeds to the next step. At this point, point-to-point coarse registration is first performed between the second feature points from the CT image (coordinates in the 3D image coordinate system of the CT image) and the first feature points from the RGB image (coordinates transformed from the 2D RGB image to the 3D camera coordinate system) to obtain a coarse registration result (i.e., the initial transformation relationship).

In operation 7, after the point-to-point coarse registration is completed, adaptive processing may be performed on regions with facial deformation. That is, using the second feature points from the CT image, the full-head reconstruction image may be cropped to retain only facial point cloud data (i.e., the second point cloud data). Using the first feature points from the RGB image, a local facial point cloud data (i.e., the first point cloud data) for the entire region from forehead to nose tip (i.e., the target portion) may be determined.

In operation 8, first fine registration may be performed between the first point cloud data from the RGB image and the second point cloud data. During the first fine registration, based on the CT image, refined zoning may be used to divide the target object's face into deformable regions (e.g., eyes, cheeks) (i.e., the second target portion) and less deformable regions (e.g., forehead, nose tip) (i.e., the first target portion), and different registration weights may be used for the first fine registration. This can improve registration accuracy for image data with facial deformations.

After the first fine registration is completed, a first fine registration result (i.e., the positioning information of the robot including a registration matrix and a registration error) may be obtained, and the registration error may be displayed. The user may determine whether to enter a second fine registration based on the display effect and the registration error.

In the second fine registration, a plurality of third images captured by the image capturing apparatus with a plurality of updated target postures may be obtained, a fusion image of the target object may be generated based on the plurality of third images, and the second fine registration may be performed based on the fusion image and the second image using the first fine registration result to obtain a second fine registration result (i.e., the updated positioning information of the robot).

It should be noted that the above descriptions of the above processes are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the above process may be accomplished with one or more additional operations not described, and/or without one or more of the operations discussed. For example, the processor 102 may further perform the registration between the target object and the planning image based on the first image, thereby improving the accuracy of the registration plan

According to some embodiments of the present disclosure, (1) by determining the first target posture of the image capturing apparatus in the base coordinate system based on the at least one target feature point and the at least one reference feature point, the image capturing apparatus can shoot the target object at the target shooting angle and/or the target shooting distance, which can result in automatic positioning of the image capturing apparatus to the optimal position, thereby improving the accuracy of the image data captured by the image capturing apparatus and the accuracy of the registration plan; (2) by capturing the first image using the adjusted image capturing apparatus, the accuracy of the first image can be improved, thereby improving the accuracy of the robot positioning; (3) by determining the transformation relationships between the coordinate systems of the robot, the image capturing apparatus, and the medical imaging device based on the target region in the first image and the second image, no additional markers need to be attached to or disposed on the target object, avoiding additional harm to the target object; (4) by performing the robot positioning based on the medical image, the accuracy of the robot positioning can be improved, thereby improving the accuracy of preoperative planning or surgical operations.

Some embodiments of the present disclosure further provide an electronic device. The electronic device includes at least one storage medium storing computer instructions, and at least one processor. When the at least one processor executes the computer instructions, the robot positioning method and the posture adjustment method of image capturing apparatus described in the present disclosure may be implemented. The electronic device may also include a transmission device and an input/output device. The transmission device and the input/output device may be connected to the at least one processor. More descriptions regarding the techniques may be found in elsewhere in the present disclosure. See, e.g., FIGS. 1A to 37, and relevant descriptions thereof.

Some embodiments of the present disclosure further provide a non-transitory computer-readable storage medium that stores the computer instruction. When reading the instruction, a computer may execute the robot positioning method and the posture adjustment method of image capturing apparatus described in the present disclosure. More descriptions regarding the techniques may be found in elsewhere in the present disclosure. See, e.g., FIGS. 1A to 37, and relevant descriptions thereof.

The basic concepts have been described above, and it will be apparent to those skilled in the art that the foregoing detailed disclosure is intended as an example only and does not constitute a limitation of the present disclosure. Although not expressly stated herein, a person skilled in the art may make various modifications, improvements, and amendments to the present disclosure. Such modifications, improvements, and amendments are suggested in the present disclosure, so such modifications, improvements, and amendments remain within the spirit and scope of the exemplary embodiments of the present disclosure.

Also, the present disclosure uses specific words to describe the embodiments of the present disclosure. For example, “an embodiment,” “one embodiment,” and/or “some embodiments” mean a feature, structure, or characteristic related to at least one embodiment of the present disclosure. Accordingly, it should be emphasized and noted that “an embodiment,” “one embodiment,” or “an alternative embodiment” referred to two or more times in different places in the present disclosure do not necessarily refer to the same embodiment. In addition, certain features, structures, or characteristics of one or more embodiments of the present disclosure may be suitably combined.

Furthermore, unless explicitly stated in the claims, the order of processing elements and sequences, the use of numerical and alphabetic characters, or the use of other names in the present disclosure are not intended to limit the sequence of the processes and methods described herein. While various examples have been discussed in the present disclosure to illustrate certain inventive embodiments that are currently considered useful, it should be understood that such details are provided for illustrative purposes and that the appended claims are not limited to the disclosed embodiments. Instead, the claims are intended to cover all modifications and equivalent combinations that fall within the spirit and scope of the embodiments described in the present disclosure. For example, while the system components described above may be implemented through hardware devices, they may also be achieved solely through software solutions, such as by installing the described system on existing servers or mobile devices.

Similarly, it should be noted that in order to simplify the presentation of the present disclosure, and thereby aid in the understanding of one or more embodiments, the preceding description of embodiments of the present disclosure sometimes incorporates a variety of features into a single embodiment, accompanying drawings, or description thereof. However, this manner of disclosure does not imply that the subject matter of the present disclosure requires more features than those mentioned in the claims. Rather, claimed subject matter may lie in less than all features of a single foregoing disclosed embodiment.

Some embodiments use numbers to describe the number of components, and attributes, and it should be understood that such numbers used in the description of the embodiments are modified in some examples by the modifiers “about,” “approximately,” or “generally.” Unless otherwise stated, “about,” “approximately,” or “generally” indicates that a variation of +20% is permitted. Accordingly, in some embodiments, the numerical parameters used in the present disclosure and claims are approximations, which may change depending on the desired characteristics of the individual embodiment. In some embodiments, the numeric parameters should be considered with the specified significant figures and be rounded to a general number of decimal places. Although the numerical domains and parameters configured to confirm the breadth of their ranges in some embodiments of the present disclosure are approximations, in specific embodiments such values are set as precisely as possible within the feasible range.

With respect to each patent, patent application, patent application disclosure, and other material, such as articles, books, manuals, publications, documents, etc., cited in the present disclosure, the entire contents thereof are hereby incorporated herein by reference. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and those described in the present disclosure, the descriptions, definitions, and/or use of terminology in the present disclosure shall prevail.

In closing, it should be understood that the embodiments described in the present disclosure are intended only to illustrate the principles of the embodiments of the present disclosure. Other deformations may also fall within the scope of the present disclosure. Thus, by way of example and not limitation, alternative configurations of embodiments of the present disclosure may be considered consistent with the teachings of the present disclosure. Accordingly, the embodiments of the present disclosure are not limited to the embodiments expressly presented and described herein.

What is claimed is:

1. A method for positioning a robot, comprising:
obtaining a target image relating to a target object, the target image being captured by an image capturing apparatus with an initial posture, the image capturing apparatus being mounted on a robot;
causing, based on the target image, the robot to adjust the image capturing apparatus to a first target posture;
obtaining a first image and a second image of the target object, the first image being captured using the image capturing apparatus with the first target posture, and the second image being captured using a medical imaging device; and
determining positioning information of the robot based on the first image and the second image.

2. The method of claim 1, wherein the first image is a depth image, and the determining positioning information of the robot based on the first image and the second image includes:
determining an initial transformation relationship between a first coordinate system corresponding to the image capturing apparatus and a third coordinate system corresponding to the medical imaging device based on a first two-dimensional (2D) reference image of the target object and the second image, the first 2D reference image being captured by the image capturing apparatus with the first target posture;
determining a second transformation relationship between the first coordinate system and the third coordinate system based on the initial transformation relationship, the first image, and the second image, and
determining the positioning information of the robot based on the second transformation relationship.

3. The method of claim 2, wherein the determining an initial transformation relationship comprises:
identifying first feature points from the first 2D reference image;
identifying second feature points from a facial reconstruction image corresponding to the second image; and
determining the initial transformation relationship by registering the first feature points and the second feature points.

4. The method of claim 3, wherein the identifying first feature points from the first 2D reference image comprises:
identifying initial feature points from the first 2D reference image;
for each initial feature point,
determining whether the initial feature point has depth information in the first image;
in response to determining that the initial feature point has depth information in the first image, designating the initial feature point as one of the first feature points; or in response to determining that the initial feature point does not have depth information in the first image, determining, based on the initial feature point and the first image, a corrected feature point as one of the first feature points.

5. The method of claim 2, wherein the determining a second transformation relationship comprises:
determining at least one first target region corresponding to at least one target portion of the target object from the first image;
determining at least one second target region corresponding to the at least one target portion from a facial reconstruction image corresponding to the second image; and
determining the second transformation relationship by registering the at least one first target region and the at least one second target region, wherein the initial transformation relationship serves as an initial value for the registration.

6. The method of claim 5, wherein the at least one target portion includes a first target portion and a second target portion, the first target portion is less affected by changes in facial expressions than the second target portion,
the registration is performed further based on a first weight value corresponding to the first target portion and a second weight value corresponding to the second target portion, and
the first weight value is greater than the second weight value.

7. The method of claim 1, further comprising:
in response to determining that the positioning information does not satisfy a preset condition,
obtaining a plurality of third images captured by the image capturing apparatus with a plurality of updated target postures;
generating a fusion image of the target object based on the plurality of third images; and
determining updated positioning information of the robot based on the fusion image and the second image.

8. The method of claim 7, wherein the obtaining a plurality of third images captured by the image capturing apparatus at a plurality of updated target postures comprises:
determining the plurality of updated target postures of the image capturing apparatus at a base coordinate system corresponding to the robot based on the second image and the positioning information; and
causing the robot to move the image capturing apparatus to the plurality of updated target postures, respectively, to obtain the plurality of third images.

9. The method of claim 1, wherein the first target posture directs the image capturing apparatus to capture the target object from a target shooting angle, and the target shooting angle is an angle directly facing the target object.

10. The method of claim 9, wherein the causing, based on the target image, the robot to adjust the image capturing apparatus to a first target posture comprises:
controlling, based on the target image, the robot to adjust the image capturing apparatus to the target shooting angle or causing a display device to display, based on the target image, guidance information for guiding a user to adjust the image capturing apparatus to the target shooting angle;
obtaining a candidate image of the target object captured by the image capturing apparatus from the target shooting angle; and
controlling, based on the candidate image, the robot to adjust the image capturing apparatus to a target shooting distance from the target object.

11. The method of claim 10, wherein the guidance information comprises at least one of:
a second 2D reference image captured by the image capturing apparatus with the initial posture;
a reference contour corresponding to the head of the target object, and
offset guidance information determined based on a current contour of the head in the second 2D reference image and the reference contour.

12. The method of claim 10, wherein the candidate image is a depth image, and the controlling, based on the candidate image, the robot to adjust the image capturing apparatus to a target shooting distance from the target object comprises:
determining whether a current shooting distance of the image capturing apparatus satisfies shooting requirements based on a third 2D reference image captured by the image capturing apparatus from the target shooting angle and the candidate image;
in response to determining that the current shooting distance of the image capturing apparatus does not satisfy shooting requirements, determining the target shooting distance based on the candidate image and causing the robot to adjust the image capturing apparatus to the target shooting distance.

13. The method of claim 12, wherein the determining whether a current shooting distance of the image capturing apparatus satisfies shooting requirements comprises:
determining third feature points in the third 2D reference image captured;
determining whether the third feature points have depth information in the candidate image;
in response to determining that one or more of the third feature points do not have depth information in the candidate image, determining that the current shooting distance does not satisfy shooting requirements.

14. The method of claim 1, wherein the first target posture directs the image capturing apparatus to capture the target object at a target shooting distance.

15. The method of claim 1, wherein the target image is an environmental image of the environment where the target object is located, and the causing, based on the target image, the robot to adjust the image capturing apparatus to a first target posture comprises:
performing target recognition on the environmental image to determine whether the target object exists in the environmental image;
in response to determining that the target object exists in the environmental image, determining the first target posture based on the environmental image and causing the robot to adjust the image capturing apparatus to the first target posture; or
in response to determining that the target object does not exist in the environmental image, guiding a user to adjust the image capturing apparatus to the first target posture.

16. The method of claim 1, wherein the determining positioning information of the robot based on the first image and the second image comprises:
performing identification on first feature points from a first 2D reference image captured by the image capturing apparatus with the first target posture;
in response to determining that the identification of one or more of the first feature points fails, presenting the first 2D reference image for guiding a user to label the first feature points on the first 2D reference image;
determining, based on the first feature points labelled by the user, at least one first target region corresponding to at least one target portion of the target object from the first image; and
determining positioning information of the robot based on the at least one first target region and the second image.

17. The method of claim 1, wherein the first target posture of the image capturing apparatus is determined based on the target image and a reference model of the target object, the reference model referring to a standard model that is constructed based on features of the target object.

18. The method of claim 1, wherein the first target posture of the image capturing apparatus is determined by:
determining a second target posture of the target object relative to the image capturing apparatus based on the initial posture;
determining a fourth transformation relationship between a first coordinate system corresponding to the image capturing apparatus and a base coordinate system corresponding to the robot; and
determining the first target posture based on the second target posture and the fourth transformation relationship.

19. A system for positioning a robot, comprising:
a storage device, configured to store a computer instruction; and
a processor connected to the storage device, wherein when executing the computer instruction, the processor causes the system to perform the following operations:
obtaining a target image relating to a target object, the target image being captured by an image capturing apparatus with an initial posture, the image capturing apparatus being mounted on a robot;
causing, based on the target image, the robot to adjust the image capturing apparatus to a first target posture;
obtaining a first image and a second image of the target object, the first image being captured using the image capturing apparatus with the first target posture, and the second image being captured using a medical imaging device; and
determining positioning information of the robot based on the first image and the second image.

20. A non-transitory computer readable medium, comprising executable instructions that, when executed by at least one processor, direct the at least one processor to perform a method, the method comprising:
obtaining a target image relating to a target object, the target image being captured by an image capturing apparatus with an initial posture, the image capturing apparatus being mounted on a robot;
causing, based on the target image, the robot to adjust the image capturing apparatus to a first target posture;
obtaining a first image and a second image of the target object, the first image being captured using the image capturing apparatus with the first target posture, and the second image being captured using a medical imaging device; and
determining positioning information of the robot based on the first image and the second image.
